(19) United States
(12) Patent Application Publication
(10) Pub. No.: US 2023/0081694 A1
(43) Pub. Date: Mar. 16, 2023

(54) ARTICLES AND METHODS FOR BLOOD SEPARATION

(71) Applicant: Trustees of Tufts College, Medford, MA (US)

(72) Inventors: Charles R. Mace, Winchester, MA (US); Keith Baillargeon, Medford, MA (US); Jessica C. Brooks, Medford, MA (US)

(73) Assignee: Trustees of Tufts College, Medford, MA (US)

(21) Appl. No.: 17/796,221

(22) PCT Filed: Jan. 29, 2021
(86) PCT No.: PCT/US2021/015624

§ 371 Date: Jul. 28, 2022

Related U.S. Application Data
(60) Provisional application No. 62/967,808, filed on Jan. 30, 2020.

(51) Int. Cl. G01N 33/49; B01D 63/08; G01N 33/50; G01N 1/40; B01L 3/00

(52) U.S. Cl. CPC G01N 33/491; B01D 63/087; G01N 33/50; G01N 1/4005; B01L 3/5023; G01N 2001/4088

(57) ABSTRACT

Disclosed herein are articles and methods for blood separation. For example, inventive articles and methods that remove red blood cells from blood samples are described.

### RELATED APPLICATIONS

This application claims priority under 35 U.S.C. § 119(e) to U.S. Provisional Patent Application No. 62/967,808, filed Jan. 30, 2020, which is hereby incorporated by reference in its entirety.

### TECHNICAL FIELD

Articles and methods for blood separation are generally described.

### SUMMARY

Disclosed herein are articles and methods for blood separation. For example, inventive articles and methods that remove red blood cells from blood samples are described. In some embodiments, the article comprises a first layer that removes red blood cells and a second layer that further removes red blood cells. In some embodiments, the first layer and/or second layer removes red blood cells with size exclusion and/or electrostatic interactions. In some embodiments, the article comprises a third layer that absorbs the purified blood (e.g., purified blood plasma). In some embodiments, the first layer, second layer, and third layer are vertically stacked. The subject matter of the present invention involves, in some cases, interrelated products, alternative solutions to a particular problem, and/or a plurality of different uses of one or more systems and/or articles.

Some embodiments relate to articles. In some embodiments, the article comprises: a first layer, wherein the first layer is porous and has a first mode pore size that is greater than or equal to 1 micron and less than or equal to 30 microns; a second layer having a first surface and a second surface, wherein the second layer is porous and greater than or equal to 20% of the pores of the second layer have a pore size of less than or equal to 20 microns; and a third layer, wherein the third layer is porous and has an absorbency of greater than or equal to 80 microliters/cm² and less than or equal to 600 microliters/cm²; and wherein the second layer is positioned between the first layer and the third layer.

Some embodiments relate to methods. In some embodiments, the method comprises: passing a blood sample across a first layer to produce a blood sample with reduced red blood cells, passing the blood sample with reduced red blood cells across a second layer to produce a blood sample with further reduced red blood cells; and passing the blood sample with further reduced red blood cells into a third layer that has an absorbency of greater than or equal to 80 microliters/cm² and less than or equal to 500 microliters/cm²; wherein the first layer, the second layer, and the third layer are porous.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is, in accordance with some embodiments, a schematic illustration of an article comprising a first layer, a second layer, and a third layer.

FIG. 2 is a schematic of a deconstructed article, according to one set of embodiments.

FIG. 3 shows a method of separating blood, according to one set of embodiments.

FIG. 4 is a plot of the recovered plasma volume as a function of separation time, according to one set of embodiments. The large plasma separation device (1.6 cm diameter) was used. The sample input volume (250 μL) and hematocrit (ca. 45%) were constant. Each data point represents the average of three replicates and error bars represent the standard error of the mean.

FIG. 5 is a bar graph showing the separation efficiency of devices of various sizes with various sample input volumes, according to one set of embodiments. The separation time (10 mins) and hematocrit (ca. 45%) were constant. Each column represents the average (N=5) and error bars represent the standard error of the mean.

FIGS. 6A-6C show a comparison of plasma quality for samples prepared using plasma separation devices (N=20), in accordance with some embodiments, or a centrifuge (N=20).

FIG. 7A is a schematic of positive (test and control lines present) and negative (only control line present) results for a tetanus lateral flow test.

FIG. 7B shows images of a reference plasma sample collected via centrifugation of whole blood (positive control), a plasma sample recovered from a plasma separation device in accordance with some embodiments (collected plasma), a plasma sample recovered from a plasma separation device in accordance with some embodiments after drying at room temperature for 16 hours and elution with buffer (rehydrated plasma), and a buffered sample without tetanus antibody (negative control).

FIG. 7C shows replicate images of lateral flow tests with plasma samples recovered from a plasma separation device in accordance with some embodiments and directly applied to the lateral flow test without centrifugation (N=5).

FIG. 8 shows the dimensions for various acrylic scaffolds, according to one set of embodiments.

FIG. 9 shows the quantitation of total protein, where FIG. 9A shows the calibration curve used and FIG. 9B shows the replicate data for plasma obtained from a device in accordance with some embodiments compared to the plasma obtained from centrifugation (N=20, p-value=0.0001).

FIG. 10 shows the calibration data for purity assessment, where FIG. 10A is a plot of four calibration curves used for h-IgG, and FIG. 10B shows the calibration plot for hemoglobin.

### DETAILED DESCRIPTION

Disclosed herein are articles and methods for blood separation. For example, inventive articles and methods that remove red blood cells from blood samples are described. In some embodiments, blood separation (e.g., removal of red blood cells from a blood sample) is desired, as removal of the cellular components (e.g., red and white blood cells) from whole blood can improve sensitivity of some clinical assays and/or reduce degradation of analytes of interest in plasma. However, this separation can be challenging, as the red blood cells in whole blood are numerous and may clog separation devices, and red blood cells are fragile and may rupture, contaminating the plasma. Moreover, this separation can be expensive, as it may require expensive high-speed centrifuges or constant manual operation, and it may produce only low volumes of plasma for large separation devices and/or long separation times. In some embodiments, the articles and/or methods described herein provide improved articles and/or methods for blood separation.

In some embodiments, the article comprises a first layer, a second layer, and a third layer. In some embodiments, the first layer is a pre-filter layer that quickly removes a significant portion of the red blood cells (and/or white blood cells) from whole blood, such that the second layer is less likely to get clogged and/or can have a higher loading capacity. In some embodiments, the second layer further removes red blood cells (and/or white blood cells). In some embodiments, the second layer has a gradient in pore size (e.g., with larger pores on the surface of the second layer adjacent to the first layer), such that the second layer is less likely to get clogged and/or is less likely to rupture the red blood cells. In some embodiments, the third layer is absorbent, so that it can absorb the purified blood. In some embodiments, the purified blood in the third layer can be used immediately (e.g., collected from and/or used directly from the third layer) or it can be stored long term (e.g., dried in the third layer). In some embodiments, the first layer, second layer, and third layer are vertically stacked.

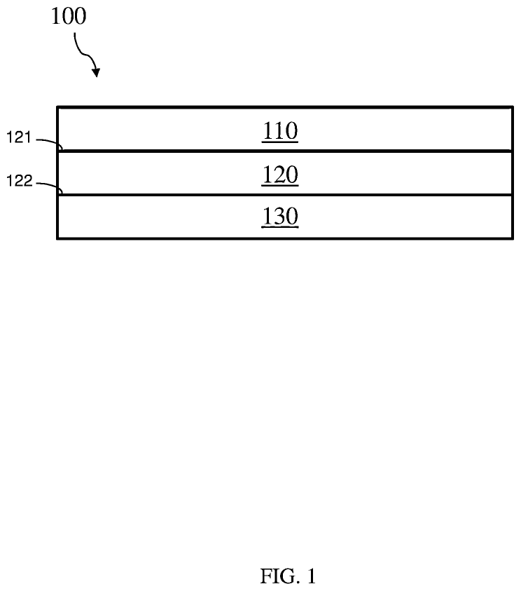

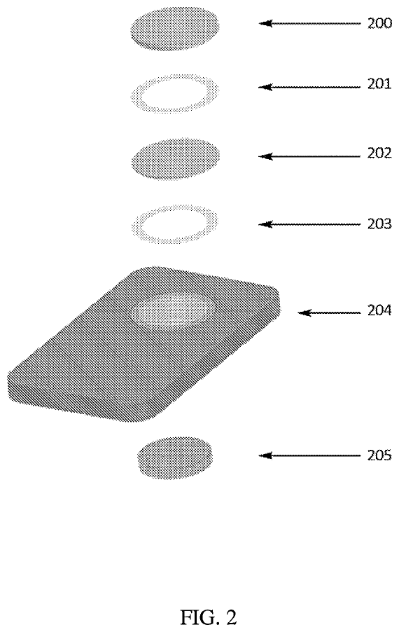

Articles are described herein. In accordance with some embodiments, articles are illustrated schematically in FIGS. 1-2.

In some embodiments, the article comprises one or more layers. In some embodiments, the article comprises greater than or equal to 1 layer, greater than or equal to 2 layers, or greater than or equal to 3 layers. In some embodiments, the article comprises less than or equal to 10 layers, less than or equal to 7 layers, less than or equal to 5 layers, less than or equal to 4 layers, or less than or equal to 3 layers. Combinations of these ranges are also possible (e.g., greater than or equal to 1 layer and less than or equal to 4 layers). In some embodiments, the article comprises a first layer, a second layer, and a third layer. For example, in some embodiments, article 100 in FIG. 1 comprises first layer 110, second layer 120, and third layer 130. Similarly, in some embodiments, the article in FIG. 2 comprises first layer 200, second layer 202, and third layer 205.

In some embodiments, the article comprises a first layer. In some embodiments, the first layer comprises a pre-filter. In some embodiments, the first layer comprises fiberglass, polyester, a fibrous membrane (e.g., polyether sulfone), and/or mesh (e.g., polyester and/or nylon). In some embodiments, the polyester comprises a treated polyester, such as Leukosorb. In some embodiments, the first layer comprises a mesh (e.g., polyester and/or nylon). In some embodiments, the first layer is treated. In some embodiments, the first layer is not treated. The first layer may be fibrous or non-fibrous.

In some embodiments, the first layer is porous. In some embodiments, the first layer has a first mode pore size. In some embodiments, the first mode pore size is greater than or equal to 1 micron, greater than or equal to 2 microns, greater than or equal to 3 microns, greater than or equal to 4 microns, greater than or equal to 5 microns, greater than or equal to 10 microns, or greater than or equal to 15 microns. In some embodiments, the first mode pore size is less than or equal to 30 microns, less than or equal to 25 microns, less than or equal to 20 microns, less than or equal to 15 microns, less than or equal to 10 microns, less than or equal to 9 microns, less than or equal to 8 microns, less than or equal to 7 microns, less than or equal to 6 microns, or less than or equal to 5 microns. Combinations of these ranges are also possible (e.g., greater than or equal to 1 micron and less than or equal to 30 microns, greater than or equal to 1 micron and less than or equal to 6 microns, greater than or equal to 2 microns and less than or equal to 25 microns, or greater than or equal to 15 microns and less than or equal to 25 microns).

In some embodiments, the first layer can have a variety of suitable thicknesses. In some embodiments, the first layer has a relatively small thickness. In some embodiments, the thickness of the first layer is greater than or equal to 150 microns, greater than or equal to 165 microns, or greater than or equal to 180 microns. In some embodiments, the thickness of the first layer is less than or equal to 500 microns, less than or equal to 400 microns, less than or equal to 300 microns, less than or equal to 250 microns, or less than or equal to 220 microns. Combinations of these ranges are also possible (e.g., greater than or equal to 180 microns and less than or equal to 220 microns, or greater than or equal to 150 microns and less than or equal to 500 microns). In some embodiments, the relatively small thickness of the first layer reduces separation time.

In some embodiments, the first layer has a relatively low absorbency. In some embodiments, the absorbency of the first layer is less than or equal to 100 microliters/cm², less than or equal to 90 microliters/cm², less than or equal to 80 microliters/cm², less than or equal to 70 microliters/cm², less than or equal to 60 microliters/cm², less than or equal to 50 microliters/cm², less than or equal to 40 microliters/cm², less than or equal to 30 microliters/cm², less than or equal to 20 microliters/cm², less than or equal to 15 microliters/cm², less than or equal to 10 microliters/cm², or less than or equal to 5 microliters/cm². In some embodiments, the absorbency of the first layer is greater than or equal to 10 microliters/cm², greater than or equal to 15 microliters/cm², greater than or equal to 20 microliters/cm², greater than or equal to 30 microliters/cm², or greater than or equal to 40 microliters/cm². Combinations of these ranges are also possible (e.g., greater than or equal to 10 microliters/cm² and less than or equal to 100 microliters/cm² or greater than or equal to 20 microliters/cm² and less than or equal to 50 microliters/cm²). In some embodiments, the relatively low absorbency of the first layer increases the separation efficiency and/or the volume of sample recovered (e.g., increases the yield of the separation), as a lower volume of the blood plasma may be retained by the first layer.

In some embodiments, the first layer comprises multiple sub-layers. For example, in some embodiments, the first layer has greater than or equal to 2 sub-layers, greater than or equal to 3 sub-layers, or greater than or equal to 4 sub-layers. In some embodiments, the first layer has less than or equal to 10 sub-layers, less than or equal to 7 sub-layers, less than or equal to 5 sub-layers, less than or equal to 4 sub-layers, less than or equal to 3 sub-layers, or less than or equal to 2 sub-layers. Combinations of these ranges are also possible (e.g., greater than or equal to 2 sub-layers and less than or equal to 10 sub-layers, or greater than or equal to 2 sub-layers and less than or equal to 4 sub-layers). In embodiments where the first layer comprises multiple sub-layers, the sub-layers may each independently have any features described herein for the first layer.

In embodiments where the first layer comprises multiple sub-layers, multiple of the sub-layers (e.g., all of the sub-layers) may comprise the same material or different material. For example, in some embodiments, the first layer comprises three sub-layers, and all of the sub-layers comprise a mesh (e.g., a polyester and/or nylon mesh). In some embodiments, one or more properties (e.g., thickness, mode pore size, mean pore size, maximum horizontal dimension, and/or absorbency) of the sub-layers (e.g., all of the sub-layers) are the same or different. In some embodiments where each of the sub-layers have a different property (e.g., mode pore size), the sub-layers are arranged such that a gradient in that property is formed. As a non-limiting example, in some embodiments, the first layer comprises three sub-layers, and each of the sub-layers has a different mode pore size such that a gradient in mode pore size is formed (e.g., 11 micron mode pore size in the first sub-layer, 6 micron mode pore size in the second sub-layer, and 1 micron mode pore size in the third sub-layer, wherein the second sub-layer is positioned between the first sub-layer and the third sub-layer).

In some embodiments, the article comprises a second layer. In some embodiments, the second layer comprises a polymer. In some embodiments, the second layer comprises polyether sulfone. In some embodiments, the second layer comprises a plasma separation membrane, such as a Pall plasma separation membrane (e.g., a Pall Vivid plasma separation membrane (e.g., grade GX and/or grade GF)), a Kinbio plasma separation membrane, and/or a Cobetter plasma separation membrane. The second layer may be fibrous or non-fibrous.

In some embodiments, the second layer is porous. In some embodiments, the second layer has a second mode pore size. In some embodiments, the second mode pore size (the mode pore size of the second layer) is greater than the first mode pore size (the mode pore size of the first layer). In some embodiments, the second mode pore size (the mode pore size of the second layer) is smaller than the first mode pore size (the mode pore size of the first layer).

In some embodiments, the second mode pore size is greater than or equal to 2 microns, greater than or equal to 3 microns, greater than or equal to 4 microns, greater than or equal to 5 microns, greater than or equal to 10 microns, or greater than or equal to 15 microns. In some embodiments, the first mode pore size is less than or equal to 30 microns, less than or equal to 25 microns, less than or equal to 20 microns, less than or equal to 15 microns, less than or equal to 10 microns, less than or equal to 9 microns, less than or equal to 8 microns, less than or equal to 7 microns, less than or equal to 6 microns, or less than or equal to 5 microns. Combinations of these ranges are also possible (e.g., greater than or equal to 2 microns and less than or equal to 30 microns or greater than or equal to 10 microns and less than or equal to 20 microns).

In some embodiments, a certain percentage of the pores of the second layer are below a certain size. In some embodiments, the certain percentage is greater than or equal to 20%, greater than or equal to 30%, greater than or equal to 40%, greater than or equal to 50%, greater than or equal to 60%, greater than or equal to 70%, greater than or equal to 80%, or greater than or equal to 90% of the pores of the second layer are below a certain size. In some embodiments, the certain percentage is less than or equal to 100%, less than or equal to 90%, less than or equal to 80%, less than or equal to 70%, less than or equal to 60%, less than or equal to 50%, less than or equal to 40%, or less than or equal to 30% of the pores of the second layer are below a certain size. Combinations of these ranges are also possible (e.g., greater than or equal to 20% and less than or equal to 100%, greater than or equal to 50% and less than or equal to 100%, or greater than or equal to 90% and less than or equal to 100%). In some embodiments, the certain size of the pores is greater than or equal to 2 microns, greater than or equal to 3 microns, greater than or equal to 4 microns, greater than or equal to 5 microns, greater than or equal to 10 microns, or greater than or equal to 15 microns. In some embodiments, the certain size of the pores is less than or equal to 30 microns, less than or equal to 25 microns, less than or equal to 20 microns, less than or equal to 15 microns, less than or equal to 10 microns, less than or equal to 9 microns, less than or equal to 8 microns, less than or equal to 7 microns, less than or equal to 6 microns, or less than or equal to 5 microns. Combinations of these ranges are also possible (e.g., greater than or equal to 2 microns and less than or equal to 30 microns or greater than or equal to 10 microns and less than or equal to 20 microns). For example, in some embodiments, greater than or equal to 20% (e.g., greater than or equal to 50% or greater than or equal to 90%) of the pores of the second layer have a pore size of less than or equal to 20 microns (e.g., greater than or equal to 10 microns and less than or equal to 20 microns).

In some embodiments, the second layer comprises a first surface and a second surface. In some embodiments, the first surface faces the first layer (e.g., is directly adjacent to a surface of the first layer). In some embodiments, the second surface faces the third layer (e.g., is directly adjacent to a surface of the third layer). For example, in some embodiments, second layer 120 in FIG. 1 comprises first surface 121, which faces first layer 110, and second surface 122, which faces third layer 130.

In some embodiments, the first surface has a mode pore size. In some embodiments, the mode pore size of the first surface is greater than or equal to 10 microns, greater than or equal to 15 microns, or greater than or equal to 20 microns. In some embodiments, the mode pore size of the first surface is less than or equal to 35 microns, less than or equal to 30 microns, or less than or equal to 25 microns. Combinations of these ranges are also possible (e.g., greater than or equal to 10 microns and less than or equal to 35 microns, greater than or equal to 15 microns and less than or equal to 25 microns, or greater than or equal to 20 microns and less than or equal to 25 microns).

In some embodiments, the second surface has a mode pore size. In some embodiments, the mode pore size of the second surface is greater than or equal to 0.01 microns, greater than or equal to 0.05 microns, greater than or equal to 0.1 microns, greater than or equal to 0.15 microns, greater than or equal to 0.25 microns, greater than or equal to 0.5 microns, or greater than or equal to 1 micron. In some embodiments, the mode pore size of the second surface is less than or equal to 5 microns, less than or equal to 3 microns, less than or equal to 1 micron, less than or equal to 0.5 microns, less than or equal to 0.3 microns, or less than or equal to 0.2 microns. Combinations of these ranges are also possible (e.g., greater than or equal to 0.01 microns and less than or equal to 1 micron, greater than or equal to 0.1 microns and less than or equal to 0.2 microns, or greater than or equal to 0.1 microns and less than or equal to 5 microns).

In some embodiments, the mode pore size of the second surface (e.g., the surface facing the third layer) is smaller than the mode pore size of the first surface (e.g., the surface facing the first layer). In some embodiments, the ratio of the mode pore size of the first surface to the mode pore size of the second surface is greater than or equal to 5:1, greater than or equal to 10:1, greater than or equal to 25:1, greater than or equal to 50:1, greater than or equal to 75:1, greater than or equal to 100:1, greater than or equal to 125:1, or greater than or equal to 150:1. In some embodiments, the ratio of the mode pore size of the first surface to the mode pore size of the second surface is less than or equal to 1,000:1, less than or equal to 500:1, less than or equal to 250:1, less than or equal to 200:1, less than or equal to 175:1, less than or equal to 150:1, less than or equal to 125:1, less than or equal to 100:1, less than or equal to 75:1, or less than or equal to 50:1. Combinations of these ranges are also possible (e.g., greater than or equal to 5:1 and less than or equal to 1,000:1, greater than or equal to 100:1 and less than or equal to 200:1, greater than or equal to 125:1 and less than or equal to 175:1, or greater than or equal to 150:1 and less than or equal to 175:1).

Mode pore size can be measured using any suitable technique. For example, in some embodiments, mode pore size can be measured using Mercury Intrusion Porosimetry or Scanning Electron Microscope (SEM). In some embodiments, mode pore size can be measured over the full thickness of the layer. In some embodiments, a layer can be divided into multiple sections along the thickness of the layer, and the mode pore size of each section can be measured.

In some embodiments, the first surface and/or the second surface each independently have a thickness that is a certain percentage of the thickness of the second layer. In some embodiments, the first surface and/or the second surface are each independently greater than or equal to 1/10 of the thickness of the second layer, greater than or equal to ⅛ of the thickness of the second layer, greater than or equal to ⅙ of the thickness of the second layer, or greater than or equal to 1/10 of the thickness of the second layer ¼ of the thickness of the second layer. In some embodiments, the first surface and/or second surface are each independently less than or equal to of the thickness of the second layer, less than or equal to ⅓ of the thickness of the second layer, less than or equal to ¼ of the thickness of the second layer, or less than or equal to ⅕ of the thickness of the second layer. Combinations of these ranges are also possible (e.g., greater than or equal to 1/10 of the thickness of the second layer and less than or equal to ½ of the thickness of the second layer, or greater than or equal to ⅛ of the thickness of the second layer and less than or equal to ¼ of the thickness of the second layer). In some embodiments, the first surface and the second surface have the same thickness.

In some embodiments, the second layer has a gradient in mode pore size between the first surface and the second surface. In some embodiments, there are cross-sections within the thickness of the second layer between the first surface and the second surface. In some embodiments the cross-sections have a mode pore size that is between the mode pore size of the first surface and the mode pore size of the second surface. For example, in that embodiment, if the mode pore size of the first surface was 11 microns and the mode pore size of the second surface was 1 micron, then the cross-sections within the thickness of the second layer between the first surface and the second surface would have mode pore sizes between 1 micron and 11 microns.

In some embodiments, the second layer can have a variety of suitable thicknesses. In some embodiments, the thickness of the second layer is greater than or equal to 100 microns. In some embodiments, the thickness of the second layer is less than or equal to 300 microns, less than or equal to 250 microns, less than or equal to 200 microns, or less than or equal to 150 microns. Combinations of these ranges are also possible (e.g., greater than or equal to 100 microns and less than or equal to 150 microns, or greater than or equal to 100 microns and less than or equal to 300 microns).

In some embodiments, the second layer has a relatively low absorbency. In some embodiments, the absorbency of the second layer is less than or equal to 50 microliters/cm², less than or equal to 40 microliters/cm², less than or equal to 30 microliters/cm², less than or equal to 25 microliters/cm², less than or equal to 20 microliters/cm², less than or equal to 15 microliters/cm², less than or equal to 10 microliters/cm², or less than or equal to 5 microliters/cm². In some embodiments, the absorbency of the second layer is greater than or equal to 10 microliters/cm², greater than or equal to 15 microliters/cm², or greater than or equal to 20 microliters/cm². Combinations of these ranges are also possible (e.g., greater than or equal to 10 microliters/cm² and less than or equal to 50 microliters/cm², or greater than or equal to 15 microliters/cm² and less than or equal to 25 microliters/cm²). In some embodiments, the relatively low absorbency of the second layer increases the separation efficiency and/or the volume of sample recovered (e.g., increases the yield of the separation), as a lower volume of the blood plasma is retained by the second layer.

In some embodiments, the article comprises a third layer. In some embodiments, the third layer comprises a wicking source. In some embodiments, the third layer comprises rayon and/or polyester (e.g., Kapmat). In some embodiments, the third layer comprises a blend of rayon and polyester, or a blend of rayon and polypropylene (e.g., ShamWow). The third layer may be fibrous or non-fibrous.

In some embodiments, the third layer is porous. In some embodiments, the third layer has a third mode pore size. In some embodiments, the third mode pore size is greater than or equal to 20 microns, greater than or equal to 30 microns, greater than or equal to 40 microns, greater than or equal to 50 microns, greater than or equal to 60 microns, greater than or equal to 70 microns, greater than or equal to 75 microns, greater than or equal to 80 microns, or greater than or equal to 90 microns. In some embodiments, the third mode pore size is less than or equal to 150 microns, less than or equal to 140 microns, less than or equal to 130 microns, less than or equal to 125 microns, less than or equal to 120 microns, less than or equal to 110 microns, or less than or equal to 100 microns. Combinations of these ranges are also possible (e.g., greater than or equal to 20 microns and less than or equal to 150 microns, greater than or equal to 75 microns and less than or equal to 125 microns, or greater than or equal to 90 microns and less than or equal to 100 microns).

In some embodiments, the third layer may have a relatively large absorbency. In some embodiments, the absorbency is greater than or equal to 55 microliters/cm², greater than or equal to 60 microliters/cm², greater than or equal to 65 microliters/cm², greater than or equal to 70 microliters/cm², greater than or equal to 75 microliters/cm², greater than or equal to 80 microliters/cm², greater than or equal to 85 microliters/cm², greater than or equal to 90 microliters/cm², greater than or equal to 95 microliters/cm², greater than or equal to 100 microliters/cm², greater than or equal to 125 microliters/cm², greater than or equal to 150 microliters/cm², greater than or equal to 175 microliters/cm², greater than or equal to 200 microliters/cm², greater than or equal to 250 microliters/cm², greater than or equal to 300 microliters/cm², or greater than or equal to 400 microliters/cm². In some embodiments, the absorbency is less than or equal to 600 microliters/cm², less than or equal to 550 microliters/cm², less than or equal to 500 microliters/cm², less than or equal to 450 microliters/cm², less than or equal to 400 microliters/cm², less than or equal to 300 microliters/cm², less than or equal to 250 microliters/cm², less than or equal to 200 microliters/cm², less than or equal to 175 microliters/cm², or less than or equal to 150 microliters/cm². Combinations of these ranges are also possible (e.g., greater than or equal to 80 microliters/cm² and less than or equal to 600 microliters/cm², greater than or equal to 100 microliters/cm² and less than or equal to 600 microliters/cm², or greater than or equal to 200 microliters/cm² and less than or equal to 450 microliters/cm²).

As used herein, the absorbency of an article and/or layer is determined by weighing the article and/or layer, saturating it in DI water for 30 seconds at room temperature, weighing it again, determining the difference between the second weight and the first weight (i.e., the weight of the DI water absorbed), and then converting this weight to a volume of water (e.g., microliters) using the density of DI water at room temperature. The volume of DI water absorbed is then normalized by dividing by the surface area (e.g., cm²) of the article and/or layer.

In some embodiments, the relatively large absorbency of the third layer facilitates passive separation by increasing capillary action and/or facilitates collection and/or storage of the absorbed fluid in the third layer.

In some embodiments, the third layer is configured to absorb a variety of suitable fluids. Examples of suitable fluids include water, blood plasma, saliva, urine, wound exudate, and/or cerebrospinal fluid. In some embodiments, the third layer is configured to absorb blood plasma.

In some embodiments, the third layer may have a relatively large release. As used herein, the release of an article and/or layer is the percentage of the absorbed water (determined as described above) that is released upon centrifugation. Once the article and/or layer is saturated in DI water for 30 seconds and the volume of DI water absorbed is calculated (as discussed above), the article and/or layer is centrifuged at an RCF of 800 g for 5 minutes. The volume of DI water released during centrifugation is then converted to a percentage of the volume of DI water that was absorbed in order to determine what percentage of the absorbed DI water was released. This value is the release of the article and/or layer.

In some embodiments, the third layer has a release that is greater than or equal to 15%, greater than or equal to 20%, greater than or equal to 25%, greater than or equal to 30%, greater than or equal to 35%, greater than or equal to 40%, greater than or equal to 60%, greater than or equal to 70%, greater than or equal to 80%, or greater than or equal to 90%. In some embodiments, the third layer has a release that is less than or equal to 100%, less than or equal to 95%, less than or equal to 90%, less than or equal to 85%, less than or equal to 80%, less than or equal to 75%, less than or equal to 70%, or less than or equal to 60%. Combinations of these ranges are also possible (e.g., greater than or equal to 35% and less than or equal to 100%, greater than or equal to 50% and less than or equal to 100%, greater than or equal to 70% and less than or equal to 100%, or greater than or equal to 70% and less than or equal to 90%).

In some embodiments, the relatively large release of the third layer increases separation efficiency and/or the volume of sample recovered (e.g., increases the yield of the separation).

In some embodiments, the third layer has a relatively large thickness (e.g., compared to the first and/or second layer(s)). In some embodiments, the thickness of the third layer is greater than or equal to 200 microns, greater than or equal to 225 microns, or greater than or equal to 250 microns. In some embodiments, the thickness of the third layer is less than or equal to 800 microns, less than or equal to 700 microns, less than or equal to 600 microns, or less than or equal to 500 microns. Combinations of these ranges are also possible (e.g., greater than or equal to 200 microns and less than or equal to 800 microns, or greater than or equal to 250 microns and less than or equal to 500 microns). In some embodiments, the relatively large thickness of the third layer increases the volume of sample recovered (e.g., increases the yield of the separation), as it increases the volume of fluid that can be absorbed.

In some embodiments, the article comprises a support structure. For example, in some embodiments, the article in FIG. 2 comprises support structure 204. In some embodiments, the support structure comprises a plastic, an acrylic, and/or a metal. In some embodiments, the support structure is a plastic scaffold or an acrylic scaffold. In some embodiments, the support structure is configured to maintain conformal contact between the third layer and one or more layers (e.g., the second layer).

In some embodiments, the support structure is adjacent one or more layers. In some embodiments, the support structure is adjacent the first layer, second layer, and/or third layer. In some embodiments, the support structure is in direct contact with one or more layers. In some embodiments, the support structure is in direct contact with the first layer, second layer, and/or third layer. In some embodiments, the support structure is in direct contact with the second layer and third layer. In some embodiments, the support structure is in direct contact with the third layer.

In some embodiments, the support structure is adhered to one or more layers (e.g., the third layer). Examples of suitable means to adhere (e.g., the support structure to one or more layers) are discussed elsewhere herein (e.g., in reference to adhering one layer to another layer). In some embodiments, the support structure is not adhered to one or more layers (e.g., not adhered to any layers). For example, in some embodiments, a portion of the article (e.g., the first layer, the second layer, and/or the third layer) sits on the support structure.

In some embodiments, the support structure comprises a cavity. In some embodiments, the cavity is used for holding a portion of the article (e.g., the first layer, the second layer, and/or the third layer). In some embodiments, the cavity is circular, oval, square, rectangular, and/or diamond shaped. In some embodiments, the cavity is of a similar shape as a cross-section (e.g., a horizontal cross-section) of a portion of the article (e.g., one or more layers, such as the third layer). For example, in some embodiments, the cavity and/or the cross-section of a portion of the article (e.g., one or more layers, such as the third layer) are both circular, oval, square, rectangular, and/or diamond shaped.

In some embodiments, the first layer, second layer, third layer, and/or article have a relatively large maximum horizontal dimension. In some embodiments, the first layer, second layer, third layer, and/or article each independently have a maximum horizontal dimension of greater than or equal to 20 millimeters, greater than or equal to 40 millimeters, greater than or equal to 60 millimeters, greater than or equal to 80 millimeters, greater than or equal to 100 millimeters, greater than or equal to 120 millimeters, greater than or equal to 140 millimeters, or greater than or equal to 150 millimeters. In some embodiments, the first layer, second layer, third layer, and/or article each independently have a maximum horizontal dimension of less than or equal to 500 millimeters, less than or equal to 400 millimeters, less than or equal to 300 millimeters, less than or equal to 200 millimeters, less than or equal to 180 millimeters, less than or equal to 160 millimeters, less than or equal to 140 millimeters, less than or equal to 120 millimeters, less than or equal to 100 millimeters, less than or equal to 80 millimeters, less than or equal to 60 millimeters, or less than or equal to 40 millimeters. Combinations of these ranges are also possible (e.g., greater than or equal to 20 millimeters and less than or equal to 500 millimeters, greater than or equal to 20 millimeters and less than or equal to 100 millimeters, greater than or equal to 60 millimeters and less than or equal to 200 millimeters). In some embodiments, the maximum horizontal dimensions of one or more (e.g., two or three) of the first layer, second layer, and third layer are the same.

In some embodiments, the relatively large maximum horizontal dimension of one or more layers (e.g., the second layer, or all of the layers) increases separation efficiency, decreases the separation time, increases the volume of sample recovered (e.g., increases the yield of the separation), and/or increases input volume.

In some embodiments, the maximum horizontal dimension of the cavity is greater than or equal to the maximum horizontal dimension of a portion of the article (e.g., one or more layers, such as the second layer and/or the third layer). In some embodiments, the ratio of the maximum horizontal dimension of the cavity to the maximum horizontal dimension of a portion of the article (e.g., one or more layers, such as the second layer and/or the third layer) is greater than or equal to 1:1, greater than or equal to 1.05:1, greater than or equal to 1.1:1, greater than or equal to 1.2:1, greater than or equal to 1.3:1, greater than or equal to 1.4:1, or greater than or equal to 1.5:1. In some embodiments, the ratio of the maximum horizontal dimension of the cavity to the maximum horizontal dimension of a portion of the article (e.g., one or more layers, such as the second layer and/or the third layer) is less than or equal to 3:1, less than or equal to 2:1, less than or equal to 1.5:1, less than or equal to 1.4:1, less than or equal to 1.3:1, less than or equal to 1.2:1, less than or equal to 1.1:1, or less than or equal to 1.05:1. Combinations of these ranges are also possible (e.g., greater than or equal to 1:1 and less than or equal to 3:1 or greater than or equal to 1.1 and less than or equal to 1.3:1).

In some embodiments, the maximum horizontal dimension of the cavity is greater than or equal to 0.5 cm, greater than or equal to 0.75 cm, greater than or equal to 1 cm, greater than or equal to 1.1 cm, greater than or equal to 1.2 cm, greater than or equal to 1.3 cm, greater than or equal to 1.4 cm, greater than or equal to 1.5 cm, greater than or equal to 1.6 cm, greater than or equal to 1.7 cm, greater than or equal to 1.8 cm, greater than or equal to 1.9 cm, greater than or equal to 2 cm, greater than or equal to 2.25 cm, greater than or equal to 2.5 cm, or greater than or equal to 3 cm. In some embodiments, the maximum horizontal dimension of the cavity is less than or equal to 10 cm, less than or equal to 5 cm, less than or equal to 4 cm, less than or equal to 3 cm, less than or equal to 2.5 cm, less than or equal to 2.25 cm, less than or equal to 2 cm, less than or equal to 1.9 cm, less than or equal to 1.8 cm, less than or equal to 1.7 cm, less than or equal to 1.6 cm, less than or equal to 1.5 cm, less than or equal to 1.4 cm, less than or equal to 1.3 cm, less than or equal to 1.2 cm, less than or equal to 1.1 cm, or less than or equal to 1 cm. Combinations of these ranges are also possible (e.g., greater than or equal to 0.5 cm and less than or equal to 10 cm or greater than or equal to 0.5 cm and less than or equal to 2 cm).

In some embodiments, the depth of the cavity is less than the thickness of the support structure, such that, when viewed from above, a layer of the support structure is present throughout the surface area of the support structure. In some embodiments, the cavity is configured such that a portion of the article (e.g., the first layer, second layer, and/or third layer) can sit inside the cavity. In some embodiments, the cavity is configured such that a portion of the article (e.g., the first layer, second layer, and/or third layer) can sit inside the cavity, with the bottom surface of the third layer in contact with the support structure.

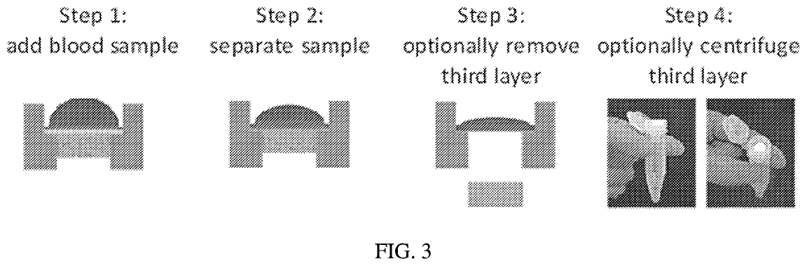

In some embodiments, the cavity is present throughout the thickness of the support structure, such that, when viewed from above, the cavity is a hole in the support structure. In some embodiments, the cavity has different maximum horizontal dimensions at different thickness of the support structure. For example, in some embodiments, the cavity has a larger maximum horizontal dimension at one opening than at the other. In some embodiments, the larger maximum horizontal dimension at one opening is greater than or equal to the maximum horizontal dimension of a portion of the article (e.g., the third layer). In some embodiments, the smaller maximum horizontal dimension at the other opening is less than the maximum horizontal dimension of a portion of the article (e.g., the third layer). In some embodiments, the cavity is configured such that a portion of the article (e.g., the first layer, second layer, and/or third layer) can sit inside the cavity. In some embodiments, the cavity is configured such that a portion of the article (e.g., the first layer, second layer, and/or third layer) can sit inside the cavity, but the bottom surface of the third layer is not in contact with the support structure. In some embodiments, the cavity is configured such that a portion of the article (e.g., the first layer, second layer, and/or third layer) can sit inside the cavity, but the bottom surface of the third layer is not in contact with the support structure, such that the third layer can be removed from the article through the bottom of the support structure (e.g., through the opening with the smaller maximum horizontal dimension), while the remaining portions of the article can remain in the support structure (see, e.g., FIG. 3).

In some embodiments, the cavity is configured such that the height of the edges (e.g., circumference) of the cavity prevent a portion of the article (e.g., the first layer, second layer, and/or third layer) from significant horizontal movement, but the portion of the article (e.g., the first layer, second layer, and/or third layer) can still be picked up vertically. In some embodiments, the height of the edges of the cavity are greater than or equal to ⅕ the thickness of a layer (e.g., the third layer), greater than or equal to ¼ the thickness of a layer (e.g., the third layer), greater than or equal to ⅓ the thickness of a layer (e.g., the third layer), greater than or equal to ½ the thickness of a layer (e.g., the third layer), or greater than or equal to the thickness of a layer (e.g., the third layer). In some embodiments, the height of the edges of the cavity are less than or equal to 3 times the thickness of a layer (e.g., the third layer), 2 times the thickness of a layer (e.g., the third layer), the thickness of a layer (e.g., the third layer), ½ the thickness of a layer (e.g., the third layer), ⅓ the thickness of a layer (e.g., the third layer), or ¼ the thickness of a layer (e.g., the third layer). Combinations of these ranges are also possible (e.g., greater than or equal to ⅕ and less than or equal to 3 times the thickness of a layer (e.g., the third layer)).

The layers in the article may be in any suitable order. In some embodiments, the first layer is positioned between the second layer and third layer. In some embodiments, the third layer is positioned between the first layer and second layer. In some embodiments, the second layer is positioned between the first layer and the third layer. For example, in FIG. 1, in accordance with some embodiments, second layer 120 is positioned between first layer 110 and third layer 130.

In some embodiments, there are no intervening layers between the first layer and second layer and/or between the second layer and third layer. For example, in FIG. 1, in accordance with some embodiments, there are no intervening layers between first layer 110 and second layer 120 or between second layer 120 and third layer 130. In some embodiments, the direct contact (e.g., direct conformal contact) between the layers (e.g., the second and third layer) decreases the separation time by increasing capillary action.

In some embodiments, one or more layers are adhered to one or more layers. For example, in some embodiments, the article in FIG. 2 comprises adhesive 201, which adheres first layer 200 to second layer 202, and adhesive 203, which adheres second layer 202 to third layer 205. In some embodiments, one or more layers are permanently adhered or integrally connected to one or more layers. In some embodiments, one or more layers are reversibly adhered to one or more layers. Examples of suitable methods of adhering layers include double-sided adhesive (e.g., double-sided medical adhesive), liquid adhesive, sonic welding, and/or compression. In some embodiments, one or more layers are adhered to one or more layers (and/or a support structure) with an adhesive. Examples of suitable adhesives include double-sided adhesive (e.g., double-side medical adhesive), compression tape, 3M brand adhesive, and/or Flexcon brand adhesive. In some embodiments, the adhesive is placed on a surface of a layer. In some embodiments, the adhesive is placed around the perimeter of a layer where it contacts another layer (or substrate) to adhere it to the other layer (or substrate). In some embodiments, the adhesive (e.g., between two layers, or between a layer and the substrate) provides a full seal (e.g., a seal around the entire perimeter of the layer through which fluid cannot pass).

In some embodiments, a full seal (e.g., with adhesive) between one or more layers (and/or between a layer and the substrate) increases the purity of the purified blood (e.g., purified plasma), as it reduces or prevent one or more impurities (e.g., red blood cells) from bypassing one or more layers and entering the third layer. For example, if there was a partial seal around the perimeter of the first layer where it contacts the second layer, then a blood sample might pass through the first layer and out through the holes in the seal, such that it then passes down to the third layer without passing through the second layer, resulting in higher levels of impurities (e.g., red blood cells) than if the blood sample had passed through the second layer.

In some embodiments, the adhesive has any suitable thickness. In some embodiments, the adhesive is relatively thin. In some embodiments, a thin adhesive allows the layers to be closer together, decreasing the separation time. In some embodiments, the adhesive has a thickness of greater than or equal to 0.03 millimeters, greater than or equal to 0.04 millimeters, greater than or equal to 0.05 millimeters, greater than or equal to 0.06 millimeters, or greater than or equal to 0.063 millimeters. In some embodiments, the adhesive has a thickness of less than or equal to 0.2 millimeters, less than or equal to less than or equal to 0.18 millimeters, less than or equal to 0.16 millimeters, less than or equal to 0.14 millimeters, or less than or equal to 0.126 millimeters. Combinations of these ranges are also possible (e.g., greater than or equal to 0.03 millimeters and less than or equal to 0.2 millimeters, or greater than or equal to 0.063 millimeters and less than or equal to 0.126 millimeters).

In some embodiments, the adhesive is applied manually. In some embodiments, the adhesive is applied with a laser cutter, ultrasonic welding, and/or UV curing. In some embodiments, the adhesive has a low tack. In some embodiments, one or more layers is adhered to one or more layers in such a way that they cannot be pulled apart manually without damaging one or more of the layers. For example, in some embodiments, the first layer is adhered to the second layer such that they cannot be pulled apart manually without damaging one or more of the layers. In some embodiments, one or more layers is adhered to one or more layers in such a way that they can be pulled apart manually without damaging one or more of the layers. For example, in some embodiments, the second layer is adhered to the third layer in such a way that they can be pulled apart manually without damaging one or more of the layers (e.g., the third layer). In some embodiments, the second layer is adhered to the third layer in such a way that they can be pulled apart manually, without having to use so much force that it will disrupt the first layer (e.g., creating mess or contamination), but such that the second layer and third layer do not come apart during use (e.g., do not come apart during separation of a blood sample).

In some embodiments, the layers are stacked coaxially, such that a vertical stack is formed. For example, in some embodiments, article 100 in FIG. 1 comprises first layer 110, second layer 120, and third layer 130 stacked coaxially, such that a vertical stack is formed. In some embodiments, the vertical stacking reduces the time required for separation.

In some embodiments, the layers described herein are discrete layers. In some embodiments, the layers described herein are not discrete layers, such that a layer is instead one of multiple phases within a discrete layer. For example, in some embodiments, the first layer and the second layer could be two phases within one layer.

In some embodiments, the maximum horizontal dimension of the article is greater than or equal to 0.5 cm, greater than or equal to 0.75 cm, greater than or equal to 1 cm, greater than or equal to 1.1 cm, greater than or equal to 1.2 cm, greater than or equal to 1.3 cm, greater than or equal to 1.4 cm, greater than or equal to 1.5 cm, greater than or equal to 1.6 cm, greater than or equal to 1.7 cm, greater than or equal to 1.8 cm, greater than or equal to 1.9 cm, greater than or equal to 2 cm, greater than or equal to 2.25 cm, greater than or equal to 2.5 cm, or greater than or equal to 3 cm. In some embodiments, the maximum horizontal dimension of the article is less than or equal to 10 cm, less than or equal to 5 cm, less than or equal to 4 cm, less than or equal to 3 cm, less than or equal to 2.5 cm, less than or equal to 2.25 cm, less than or equal to 2 cm, less than or equal to 1.9 cm, less than or equal to 1.8 cm, less than or equal to 1.7 cm, less than or equal to 1.6 cm, less than or equal to 1.5 cm, less than or equal to 1.4 cm, less than or equal to 1.3 cm, less than or equal to 1.2 cm, less than or equal to 1.1 cm, or less than or equal to 1 cm. Combinations of these ranges are also possible (e.g., greater than or equal to 0.5 cm and less than or equal to 5 cm or greater than or equal to 0.5 cm and less than or equal to 2 cm).

In some embodiments, the article has a high loading capacity (e.g., for whole blood). As used herein, loading capacity is defined as volume of fluid that can be loaded divided by the surface area of the article. In some embodiments, the loading capacity of the article is greater than or equal to 20 microliters/cm², greater than or equal to 30 microliters/cm², greater than or equal to 40 microliters/cm², greater than or equal to 50 microliters/cm², greater than or equal to 60 microliters/cm², greater than or equal to 70 microliters/cm², greater than or equal to 80 microliters/cm², greater than or equal to 90 microliters/cm², greater than or equal to 100 microliters/cm², or greater than or equal to 125 microliters/cm². In some embodiments, the loading capacity of the article is less than or equal to 500 microliters/cm², less than or equal to 400 microliters/cm², less than or equal to 300 microliters/cm², less than or equal to 250 microliters/cm², less than or equal to 200 microliters/cm², less than or equal to 150 microliters/cm², less than or equal to 125 microliters/cm², less than or equal 100 microliters, less than or equal 90 microliters/cm², less than or equal 80 microliters/cm², or less than or equal 70 microliters/cm². Combinations of these ranges are also possible (e.g., greater than or equal to 20 microliters/cm² and less than or equal to 500 microliters/cm², or greater than or equal to 50 microliters/cm² and less than or equal to 150 microliters/cm²).

Methods are described herein. In accordance with some embodiments, an illustrative method is illustrated schematically in FIG. 3, and can be understood in view of FIG. 1.

In some embodiments, the method comprises passing a blood sample across a first layer. For example, in some embodiments, the method comprises passing a blood sample across first layer 110 in FIG. 1. In some embodiments, the first layer comprises any embodiment of the first layer, or combinations thereof, disclosed herein.

In some embodiments, the blood sample is whole blood. In some embodiments, the blood sample is diluted with water and/or a buffer solution. In some embodiments, the blood sample is undiluted blood from a subject. In some embodiments, the subject is an animal, such as a mammal. In some embodiments, the subject is a human. In some embodiments, the article comprises an anti-coagulant (e.g., ethylenediaminetetraacetic acid (EDTA) and/or heparin), such as a dried anti-coagulant.

In some embodiments, the first layer has a high loading capacity, such that the blood sample passed across the first layer (e.g., input volume) has a substantial volume. In some embodiments, the volume of the blood sample passed across the first layer (e.g., input volume) is greater than or equal to 25 microliters, greater than or equal to 30 microliters, greater than or equal to 40 microliters, greater than or equal to 50 microliters, greater than or equal to 60 microliters, greater than or equal to 70 microliters, greater than or equal to 80 microliters, greater than or equal to 90 microliters, greater than or equal to 100 microliters, greater than or equal to 125 microliters, greater than or equal to 150 microliters, greater than or equal to 200 microliters, or greater than or equal to 250 microliters. In some embodiments, the volume of the blood sample passed across the first layer (e.g., input volume) is less than or equal to 500 microliters, less than or equal to 400 microliters, less than or equal to 300 microliters, less than or equal to 250 microliters, less than or equal to 200 microliters, less than or equal to 150 microliters, less than or equal to 125 microliters, less than or equal 100 microliters, less than or equal 90 microliters, less than or equal 80 microliters, or less than or equal 70 microliters. Combinations of these ranges are also possible (e.g., greater than or equal to 25 microliters and less than or equal to 500 microliters, greater than or equal to 50 microliters and less than or equal to 300 microliters, or greater than or equal to 100 microliters and less than or equal to 250 microliters).

In some embodiments, the volume of the blood sample passed across the first layer (e.g., input volume) may affect the volume of sample (e.g., plasma) recovered, the separation efficiency, the separation time, and/or the purity (e.g., levels of hemolysis) of the sample (e.g., plasma). For example, if the volume of the blood sample passed across the first layer (e.g., input volume) is too low, then a larger percentage of the blood sample may be absorbed by the first layer and/or second layer resulting in low volume of sample recovered (e.g., low yield of the separation) and/or low separation efficiency compared to if a larger volume of the blood sample passed across the first layer (e.g., input volume), in some embodiments. As another example, if the volume of the blood sample passed across the first layer (e.g., input volume) is too high, then one or more layers may clog, resulting in more impurities passing through, increased hemolysis, and/or decreased separation time, in some embodiments.

In some embodiments, passing the blood sample across the first layer produces a blood sample with reduced red blood cells. In some embodiments, the red blood cells are reduced by the first layer by greater than or equal to 20%, greater than or equal to 30%, greater than or equal to 40%, greater than or equal to 50%, greater than or equal to 60%, greater than or equal to 70%, greater than or equal to 80%, or greater than or equal to 90% of those in the blood sample. In some embodiments, the red blood cells are reduced by the first layer by less than or equal to 100%, less than or equal to 90%, less than or equal to 80%, less than or equal to 70%, less than or equal to 60%, less than or equal to 50%, less than or equal to 40%, or less than or equal to 30% of those in the blood sample. Combinations of these ranges are also possible (e.g., greater than or equal to 20% and less than or equal to 90%).

In some embodiments, the first layer reduces the level of red blood cells in the blood sample by size exclusion and/or electrostatic interactions.

In some embodiments, the first layer reduces the level of white blood cells (which can also be called “leukocytes”). In some embodiments, the white blood cells are reduced by the first layer by greater than or equal to 20%, greater than or equal to 30%, greater than or equal to 40%, greater than or equal to 50%, greater than or equal to 60%, greater than or equal to 70%, greater than or equal to 80%, or greater than or equal to 90% of those in the blood sample. In some embodiments, the white blood cells are reduced by the first layer by less than or equal to 100%, less than or equal to 90%, less than or equal to 80%, less than or equal to 70%, less than or equal to 60%, less than or equal to 50%, less than or equal to 40%, or less than or equal to 30% of those in the blood sample. Combinations of these ranges are also possible (e.g., greater than or equal to 20% and less than or equal to 90%).

In some embodiments, the first layer reduces the level of white blood cells in the blood sample by size exclusion, electrostatic interactions, and/or adsorption of the white blood cells.

In some embodiments, use of the first layer facilitates quick removal of a significant portion of the red blood cells (and/or white blood cells), such that the second layer is less likely to get clogged and/or is less likely to cause hemolysis and/or the article can have a higher loading capacity without requiring lengthy times for separation.

In some embodiments, the method comprises passing the blood sample with reduced red blood cells (and/or white blood cells) across a second layer. For example, in some embodiments, the method comprises passing the blood sample with reduced red blood cells (and/or white blood cells) across second layer 120 in FIG. 1. In some embodiments, the second layer comprises any embodiment of the second layer, or combinations thereof, disclosed herein.

In some embodiments, passing the blood sample with reduced red blood cells (and/or white blood cells) across the second layer produces a blood sample with further reduced red blood cells. In some embodiments, the red blood cells are reduced by the second layer by greater than or equal to 20%, greater than or equal to 30%, greater than or equal to 40%, greater than or equal to 50%, greater than or equal to 60%, greater than or equal to 70%, greater than or equal to 80%, or greater than or equal to 90% of those in the blood sample with reduced red blood cells. In some embodiments, the red blood cells are reduced by the second layer by less than or equal to 100%, less than or equal to 90%, less than or equal to 80%, less than or equal to 70%, less than or equal to 60%, less than or equal to 50%, less than or equal to 40%, or less than or equal to 30% of those in the blood sample with reduced red blood cells. Combinations of these ranges are also possible (e.g., greater than or equal to 20% and less than or equal to 90%).

In some embodiments, the second layer further reduces the level of red blood cells in the blood sample with reduced red blood cells (and/or white blood cells) by size exclusion and/or electrostatic interactions.

In some embodiments, the second layer reduces the level of white blood cells. In some embodiments, the white blood cells are reduced by the second layer by greater than or equal to 20%, greater than or equal to 30%, greater than or equal to 40%, greater than or equal to 50%, greater than or equal to 60%, greater than or equal to 70%, greater than or equal to 80%, or greater than or equal to 90% of those in the blood sample with reduced red blood cells. In some embodiments, the white blood cells are reduced by the second layer by less than or equal to 100%, less than or equal to 90%, less than or equal to 80%, less than or equal to 70%, less than or equal to 60%, less than or equal to 50%, less than or equal to 40%, or less than or equal to 30% of those in the blood sample with reduced red blood cells. Combinations of these ranges are also possible (e.g., greater than or equal to 20% and less than or equal to 90%).

In some embodiments, the second layer reduces the level of white blood cells in the blood sample with reduced red blood cells by size exclusion and/or electrostatic interactions.

In some embodiments, use of a second layer with a gradient in pore size reduces the risk of the second layer clogging and/or reduces the risk that the second layer will result in hemolysis, in some embodiments.

In some embodiments, the method comprises passing the blood sample with further reduced red blood cells into a third layer. For example, in some embodiments, the method comprises passing a blood sample with further reduced red blood cells into third layer 130 in FIG. 1. In some embodiments, the third layer comprises any embodiment of the third layer, or combinations thereof, disclosed herein.

In some embodiments, the method (e.g., passing the blood sample across the first layer, passing the blood sample with reduced red blood cells across the second layer, and/or passing the blood sample with further reduced red blood cells into the third layer) is passive. For example, in some embodiments, the method is done solely with the use of gravity and/or capillary action. In some embodiments, the method is done without the use of centrifugation, electricity, and/or an external field (e.g., acoustic, electric, and/or magnetic). For example, in some embodiments, FIG. 3 demonstrates adding blood sample to the article (e.g., the first layer) and then the article separates the sample without further action (that is, the sample is separated purely from gravity and capillary action).

In some embodiments, a portion of the method (e.g., passing the blood sample across the first layer, passing the blood sample with reduced red blood cells across the second layer, and/or passing the blood sample with further reduced red blood cells into the third layer) is relatively rapid as the separation time is short. In some embodiments, a portion of the method is accomplished within (and/or the separation time is) less than or equal to 30 minutes, less than or equal to 20 minutes, less than or equal to 15 minutes, less than or equal to 10 minutes, less than or equal to 5 minutes, less than or equal to 3 minutes, or less than or equal to 2 minutes. In some embodiments, a portion of the method is accomplished within (and/or the separation time is) greater than or equal to 30 seconds, greater than or equal to 1 minute, greater than or equal to 2 minutes, greater than or equal to 3 minutes, or greater than or equal to 5 minutes. Combinations of these ranges are also possible (e.g., greater than or equal to 30 seconds and less than or equal to 10 minutes or greater than or equal to 30 seconds and less than or equal to 5 minutes).

In some embodiments, the method (e.g., passing the blood sample across the first layer, passing the blood sample with reduced red blood cells across the second layer, and/or passing the blood sample with further reduced red blood cells into the third layer) has a high separation efficiency. In some embodiments, the separation efficiency is greater than or equal to 10%, greater than or equal to 15%, greater than or equal to 20%, greater than or equal to 25%, greater than or equal to 30%, greater than or equal to 35%, greater than or equal to 40%, greater than or equal to 45%, greater than or equal to 50%, or greater than or equal to 55%. In some embodiments, the separation efficiency is less than or equal to 100%, less than or equal to 90%, less than or equal to 80%, less than or equal to 70%, less than or equal to 60%, less than or equal to 55%, less than or equal to 50%, less than or equal to 45%, less than or equal to 40%, less than or equal to 35%, or less than or equal to 30%. Combinations of these ranges are also possible (e.g., greater than or equal to 10% and less than or equal to 100%, greater than or equal to 10% and less than or equal to 60%, or greater than or equal to 30% and less than or equal to 55%).

As used herein, the separation efficiency is the percentage of collected purified plasma volume (or volume of purified plasma that passes into the third layer) compared to the total theoretical plasma volume. The total theoretical plasma volume is based on the measured hematocrit value and input sample volume. For example, if a 100 microliter sample has a measured hematocrit value of 50%, then the total theoretical plasma volume is 50 microliters. If 40 microliters of purified plasma were collected (or passed into the third layer), the separation efficiency would be 80%, since 40 microliters is 80% of 50 microliters.

In some embodiments, the method comprises removing the third layer from the second layer. For example, in some embodiments, FIG. 3 demonstrates removing the third layer from the second layer. In some embodiments, the third layer is removed from the second layer by pulling it apart from the second layer. In some embodiments, the third layer is pulled apart from the second layer manually (e.g., pulling it apart with tweezers). In some embodiments, the article comprises a tab. In some embodiments, pulling the tab may pull the third layer apart from the second layer.

In some embodiments, the blood sample with further reduced red blood cells is used directly from the third layer. For example, in some embodiments, the third layer can be used as a stamp with which to apply the blood sample with further reduced red blood cells (e.g., to a lateral flow test).

In some embodiments, the blood sample with further reduced red blood cells is stored inside the third layer. In some embodiments, the blood sample with further reduced red blood cells is stored inside the third layer in a wet state. In some embodiments, the blood sample with further reduced red blood cells is stored inside the third layer in a dry state. For example, in some embodiments, the third layer containing the blood sample with further reduced red blood cells is dried overnight. In some embodiments, the third layer is dried overnight in a sealed container. In some embodiments, the sealed container comprises a desiccant.

In some embodiments, the dried third layer is later rehydrated. In some embodiments, the dried third layer is rehydrated by adding a solvent, such as an aqueous solution (e.g., an aqueous solution comprising a surfactant), a buffered solution (e.g., phosphate buffered saline), and/or water (e.g., DI water).

In some embodiments, the method comprises collecting the blood sample with further reduced red blood cells from the third layer. In some embodiments, collecting the blood sample with further reduced red blood cells is done shortly after the blood sample with further reduced red blood cells is passed into the third layer. In some embodiments, collecting the blood sample with further reduced red blood cells is done after the sample with further reduced blood cells has been stored (e.g., in a wet state or in a dry state) inside the third layer for a length of time. In some embodiments, the blood sample with further reduced red blood cells is collected from the third layer greater than or equal to 1 minute, greater than or equal to 5 minutes, greater than or equal to 15 minutes, greater than or equal to 30 minutes, greater than or equal to 1 hour, greater than or equal to 5 hours, greater than or equal to 12 hours, greater than or equal to 1 day, greater than or equal to 3 days, greater than or equal to 1 week, greater than or equal to 1 month, greater than or equal to 6 months, or greater than or equal to 1 year after it has been passed into the third layer. In some embodiments, the blood sample with further reduced red blood cells is collected from the third layer less than or equal to 3 years, less than or equal to 2 years, less than or equal to 1 year, less than or equal to 6 months, less than or equal to 1 month, less than or equal to 1 week, less than or equal to 3 days, less than or equal to 1 day, less than or equal to 12 hours, less than or equal to 5 hours, less than or equal to 1 hour, less than or equal to 30 minutes, less than or equal to 15 minutes, or less than or equal to 5 minutes after it has been passed into the third layer. Combinations of these ranges are also possible (e.g., greater than or equal to 1 minute and less than or equal to 3 years).

In some embodiments, collecting the blood sample with further reduced red blood cells from the third layer can be accomplished with relatively low amounts of force. In some embodiments, collecting the blood sample with further reduced red blood cells comprises compression (e.g., squeezing) and/or centrifuging the third layer (e.g., with a benchtop centrifuge). For example, in some embodiments, FIG. 3 demonstrates collecting the blood sample with further reduced red blood cells from the third layer by centrifugation with a benchtop centrifuge. In some embodiments, the blood sample is centrifuged at less than or equal to 800×g (e.g., less than or equal to 700×g, less than or equal to 500×g, or less than or equal to 300×g) for less than or equal to 5 minutes (e.g., less than or equal to 4 minutes, less than or equal to 3 minutes, less than or equal to 2 minutes, or less than or equal to 1 minute).

In some embodiments, the blood sample with further reduced red blood cells (e.g., the blood sample with further reduced red blood cells collected from the third layer) can be collected in a short period of time. In some embodiments, the blood sample with further reduced blood cells can be collected in less than or equal to 30 minutes, less than or equal to 20 minutes, less than or equal to 15 minutes, less than or equal to 10 minutes, less than or equal to 5 minutes, less than or equal to 3 minutes, or less than or equal to 1 minute. In some embodiments, the blood sample with further reduced blood cells can be collected in greater than or equal to 30 seconds, greater than or equal to 1 minute, greater than or equal to 2 minutes, greater than or equal to 3 minutes, or greater than or equal to 5 minutes. Combinations of these ranges are also possible (e.g., greater than or equal to 30 seconds and less than or equal to 30 minutes, or greater than or equal to 30 seconds and less than or equal to 10 minutes).

In some embodiments the blood sample with further reduced red blood cells (e.g., pure plasma) can be used in subsequent applications (e.g., after collection, and/or directly, from the third layer), such as in a diagnostic health test, a clinical assay (e.g., clinical chemistry assays), an immunoassay, an immunochromatographic assay for antibodies (e.g., tetanus antibodies), quantification of cytokines, amplification of viral RNA, a rapid dipstick test, an HIV viral load assay, a cholesterol test, a metabolite panel, serology for infectious diseases, therapeutic drug monitoring, an ELISA, ICP-AES, HPLC, and/or mass spectrometry.

In some embodiments, the volume of the blood sample with further reduced red blood cells (e.g., the blood sample with further reduced red blood cells collected and/or used directly from the third layer) is a significant percentage of the volume of the blood sample (e.g., the blood sample passed through the first layer), given that 20-60% of the blood sample (e.g., whole blood) is expected to be red blood cells. In some embodiments, the volume of the blood sample with further reduced red blood cells is greater than or equal to 10%, greater than or equal to 12%, greater than or equal to 15%, greater than or equal to 17%, greater than or equal to 20%, greater than or equal to 25%, greater than or equal to 30%, greater than or equal to 35%, greater than or equal to 40%, greater than or equal to 45%, or greater than or equal to 50% of the volume of the blood sample. In some embodiments, the volume of the blood sample with further reduced red blood cells is less than or equal to 80%, less than or equal to 70%, less than or equal to 60%, less than or equal to 50%, less than or equal to 40%, less than or equal to 30%, less than or equal to 25%, less than or equal to 20%, less than or equal to 17%, or less than or equal to 15% of the volume of the blood sample. Combinations of these ranges are also possible (e.g., greater than or equal to 10% and less than or equal to 80% or greater than or equal to 10% and less than or equal to 40%).

In some embodiments, a large volume of the blood sample with further reduced red blood cells is passed into the third layer and/or a large volume of the blood sample with further reduced red blood cells is collected and/or used directly from the third layer. For example, in some embodiments, the volume of the blood sample with further reduced red blood cells passed into the third layer and/or collected and/or used directly from the third layer is greater than or equal to 20 microliters, greater than or equal to 25 microliters, greater than or equal to 30 microliters, greater than or equal to 35 microliters, greater than or equal to 40 microliters, greater than or equal to 45 microliters, greater than or equal to 50 microliters, greater than or equal to 55 microliters, greater than or equal to 60 microliters, greater than or equal to 65 microliters, or greater than or equal to 70 microliters. In some embodiments, the volume of the blood sample with further reduced red blood cells passed into the third layer and/or collected and/or used directly from the third layer is less than or equal to 150 microliters, less than or equal to 125 microliters, less than or equal to 100 microliters, less than or equal to 90 microliters, less than or equal to 80 microliters, less than or equal to 75 microliters, less than or equal to 70 microliters, or less than or equal to 60 microliters. Combinations of these ranges is also possible (e.g., greater than or equal to 20 microliters and less than or equal to 150 microliters, greater than or equal to 30 microliters and less than or equal to 150 microliters, greater than or equal to 50 microliters and less than or equal to 150 microliters, or greater than or equal to 50 microliters and less than or equal to 100 microliters).

In some embodiments, the blood sample with further reduced red blood cells (e.g., the blood sample with further reduced red blood cells collected and/or used directly from the third layer or the blood sample) is pure (e.g., pure plasma and/or serum), substantially free of red blood cells, and/or substantially free of white blood cells. In some embodiments, the blood sample with further reduced red blood cells has less than or equal to 5%, less than or equal to 4%, less than or equal to 3%, less than or equal to 2%, or less than or equal to 1% of the red blood cells in the blood sample (e.g., the original blood sample, such as a whole blood sample). In some embodiments, the blood sample with further reduced red blood cells has less than or equal to 5%, less than or equal to 4%, less than or equal to 3%, less than or equal to 2%, or less than or equal to 1% of the white blood cells in the blood sample (e.g., the original blood sample, such as a whole blood sample).

In some embodiments, the amount of red blood cells is assumed to be the same as the amount of hemoglobin. For example, if a blood sample (e.g., an original blood sample, such as a whole blood sample) had 12 g/dL hemoglobin, and the blood sample with further reduced red blood cells has 0.12 g/dL hemoglobin, then the blood sample with further reduced red blood cells has less than or equal to 1% of the hemoglobin in the original sample, and it would be assumed that the blood sample with further reduced red blood cells has less than or equal to 1% of the red blood cells in the blood sample (e.g., the original blood sample, such as a whole blood sample).

In some embodiments, the blood sample with further reduced red blood cells (e.g., the blood sample with further reduced red blood cells collected and/or used directly from the third layer) has minimal amounts of hemolysis. In some embodiments, the blood sample with further reduced red blood cells has less than or equal to 15% hemolysis, less than or equal to 10% hemolysis, less than or equal to 8% hemolysis, less than or equal to 7%, less than or equal to 6%, less than or equal to 5% hemolysis, less than or equal to 3% hemolysis, less than or equal to 2% hemolysis, or less than or equal to 1% hemolysis. In some embodiments, the blood sample with further reduced red blood cells has greater than or equal to 0% hemolysis, greater than or equal to 0.1% hemolysis, greater than or equal to 0.5% hemolysis, greater than or equal to 1% hemolysis, greater than or equal to 2% hemolysis, greater than or equal to 3% hemolysis, greater than or equal to 4%, or greater than or equal to 5% hemolysis. Combinations of these ranges are also possible (e.g., greater than or equal to 0% and less than or equal to 15% or greater than or equal to 0.1% and less than or equal to 7%).

As used herein, the percentage hemolysis is the percentage of hemoglobin in the measured sample compared to hemoglobin in a similar whole blood sample. For example, if a blood sample was divided in two, and one part was purified (e.g., separated from red blood cells) while the other part was untreated, the percentage hemolysis in the purified sample would be the percentage of hemoglobin in that sample compared to the percentage hemoglobin in the untreated whole blood sample. The amount of hemoglobin can be measured by any suitable assay. For example, the amount of hemoglobin can be measured by the assay described in the example, where a ratio of whole blood (the control) to Drabkin's reagent containing 0.05% (v/v) Brij 25 was 1:250; a ratio of sample to Drabkin's reagent containing 0.05% (v/v) Brij 25 was 1:10; calibration curves were prepared daily using lyophilized hemoglobin standard rehydrated with diH2O (18 MΩ) and diluted over a range 3-20 g/dL; samples were incubated at 21° C. for 15 minutes and absorbance was measured at 540 nm using a microplate reader (e.g., Varioskan LUX).

In some embodiments, the blood sample with further reduced red blood cells (e.g., the blood sample with further reduced red blood cells collected and/or used directly from the third layer) has similar levels of an analyte of interest as the original blood sample (e.g., whole blood and/or the blood sample passed across the first layer). For example, in some embodiments, the level of an analyte of interest in the blood sample with further reduced red blood cells is greater than or equal to 40%, greater than or equal to 45%, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 98%, or greater than or equal to 99% the level of the analyte of interest in the original blood sample (e.g., whole blood and/or the blood sample passed across the first layer). In some embodiments, the level of an analyte of interest in the blood sample with further reduced red blood cells is less than or equal to 100%, less than or equal to 99%, less than or equal to 98%, less than or equal to 95%, less than or equal to 90%, less than or equal to 85%, less than or equal to 80%, less than or equal to 75%, or less than or equal to 70% the level of the analyte of interest in the original blood sample (e.g., whole blood and/or the blood sample passed across the first layer). Combinations of these ranges are also possible (e.g., greater than or equal to 40% and less than or equal to 100% or greater than or equal to 80% and less than or equal to 100%). For example, if a 250 microliter sample of whole blood tested for the presence of HIV RNA by RT-qPCR had an average threshold cycle value of 28 Ct and was passed across an article described herein (e.g., passed across a first layer, passed across a second layer, and passed into a third layer) to form 60 microliters of a blood sample with further reduced red blood cells (e.g., as in a method described herein) with an average threshold cycle value of 29 Ct, then the level of HIV RNA in the blood sample with further reduced red blood cells would be 50% of that in the original blood sample, as every 1 Ct in qPCR is responsible for a doubling.

Examples of analytes of interest may include proteins (e.g., enzymes (e.g., alkaline phosphatase, alanine aminotransferase, aspartate aminotransferase), antibodies (e.g., for immune response (e.g., acute IgM or persistent IgG), such as to indicate vaccination (e.g., measles), infection (e.g., HIV, SARS-CoV-2, tuberculosis, sexually transmitted infections), sensitivity to foods, allergens), and/or biomarkers (e.g., HbA1c, albumin, insulin, cancer antigens (PSA, CA-125))), nucleic acids (e.g., recovered from pathogens (e.g., RNA or DNA genes), host cell genome (e.g., to determine mutations), or cell free fetal DNA (cffDNA)), pathogens (e.g., viruses (e.g., HIV), parasites (e.g., P. falciparum), and/or bacteria (e.g., S. aureus)), metabolites (e.g., blood urea nitrogen, creatinine, bilirubin, carnosine, UDP-acetyl-glucosamine), hormones (e.g., thyroid, fertility/pregnancy, testosterone, cortisol), electrolytes (e.g., calcium, potassium, bicarbonate, chloride), lipids (e.g., HDL, LDL, VLDL, cholesterol, triglycerides), and/or small molecules (e.g., vitamins (e.g., folic acid, B vitamins, biotin) and/or sugars (e.g., glucose, Carbohydrate antigen 19-9 (sialyl-Lewis^{A}), sialyl-LewisX)).

In some embodiments, the method may be performed on any embodiment of the article, or combinations thereof, disclosed herein. In some embodiments, the article is configured to perform any embodiment of the method, or combinations thereof, disclosed herein.

In some, but not all, embodiments, the article and/or method has one or more advantages, such as short separation time, short collection time, ease of separation (e.g., without constant manual operation), ease of collection (e.g., without the use of high speed centrifuges), small surface area (e.g., small maximum horizontal dimension) of the article, ease of scaling up, ease of storage of the purified sample, large loading capacity, large volume recovery, low amounts of clogging of the article, low amounts of hemolysis in the recovered sample, high purity of the recovered sample, low amounts of mess (e.g., high containment of the blood within the article), low energy requirements, and/or ability to use whole blood samples without the need for dilution.

The following examples are intended to illustrate certain embodiments of the present invention, but do not exemplify the full scope of the invention.

### EXAMPLES

Described herein is, in accordance with some embodiments, an assembly of porous materials capable of obtaining high volumes (>60 μL) of pure plasma from whole blood using only passive methods in less than 10 minutes.

A pre-filter material was used to reduce the burden of excess blood cells from clogging the plasma separation membrane and minimize hemolysis independent of hematocrit. Separation and collection were facilitated by a super absorbent material in direct contact with the plasma separation membrane. The dual functionality of the collection pad permitted storage of purified plasma for shipping and future laboratory analysis similar to dried blood spot card technologies. The purity of collected plasma samples was evaluated by quantification of hemoglobin and the recovery of high and low concentration analytes of interest was evaluated.

### Experimental Design

### Device Design and Fabrication

The device comprised a pre-filter material, plasma separation membrane (PSM), and super absorbent material (FIG. 2).

The separation materials (e.g., pre-filter and plasma separation membrane) were affixed to the acrylic scaffold via rings of double-sided medical adhesive. The absorbent material was located in direct contact with the underside of the plasma separation membrane. Contact between each material was maintained by an acrylic scaffold and double-sided medical adhesive. The pre-filter material was designed to remove white blood cells from the sample matrix based on size exclusion and electrostatic interactions. The plasma separation membrane was designed to exclude all remaining white and red blood cells to produce pure plasma that can be simultaneously collected and stored by the underlying absorbent material.

All porous materials (e.g., pre-filter materials, PSM, and absorbent materials) were cut using a hammer-driven hole punch. Double-sided medical adhesive was patterned into rings using an automated knife plotter. Acrylic scaffolds were fabricated with a Trotech laser cutter.

The pore sizes of several materials evaluated for the device are shown in Table 1.

TABLE 1
Pore data for various materials.

| | | Surface | Mode pore | Unique pore |
|---|---|---|---|---|
| | | area | diameter | diameters |
| Sample ID | Material | (m²/g) | (μm) | (μm) |
| TFN | Cellulose | 9.8 | 27.9 | 27.9, 19.8, 9.0 |
| Kapmat | Polyester | 17.1 | 159.4 | 159.4 |
| ShamWow | Rayon/ | 32.2 | 91.6 | 91.6 |
| | Polypropylene | | | |
| | Blend | | | |
| PSM-GR | Polysulfone | 43.7 | 16.3 | 23.9, 16.3, |
| | | | | 10.0, 7.4, 4.5, |
| | | | | 0.15 |
| Leukosorb | Polyester | 43.7 | 19.5 | 19.5 |

### Operation of the Device

Plasma separation was initiated by applying a sample of whole blood to the top of the device and allowing it to sit for 5-10 minutes for separation to occur (see the schematic in FIG. 3). Purified plasma was collected by the absorbent material located beneath the plasma separation membrane. To terminate separation, the absorbent material was removed from the acrylic scaffold with a pair of tweezers and either (i) liquid plasma was recovered from the absorbent material via centrifugation, (ii) the porous material containing purified plasma was dried and stored for future laboratory analysis, or (iii) the absorbent material was immediately applied to a lateral flow test.

### Evaluation of Absorbency and Release for Porous Materials

The absorbency and release for the materials was determined as follows. The initial mass of each absorbent material was recorded (N=3, area=1 cm²). Then, each material was saturated in deionized water for 30 seconds and the saturated mass was recorded. The volume of water absorbed by each material was calculated using the density of water at ambient temperature. This value was normalized by the surface area of the material. This normalized value represented the “absorbency” of the material.

Then, the saturated absorbent materials were centrifuged to collect the water using a Swinex funnel attached to a 5-mL Eppendorf tube at an RCF of 800 g for 5 minutes. The Eppendorf tube was weighed empty and then with the released water, and the volume of water released by each material was calculated using the density of water at ambient temperature. This value represented the volume recovery. This volume was converted to a percentage of the water that was absorbed, and this value represented the “release” of the material.

### Quantification of the Recovered Plasma Volume and Calculation of Separation Efficiency

A centrifuge was used to quantify the volume of plasma collected in the devices as proof-of-concept (see, e.g., the schematic shown in FIG. 3). After plasma separation occurred, the absorbent material was removed from the acrylic scaffold using tweezers and added to a Swinex funnel attached to a 5-mL Eppendorf tube. The samples were centrifuged at an RCF of 800 g for 5 minutes to collect liquid plasma from the absorbent material. The mass of the liquid plasma was determined by calculating the difference between the initial mass of the 5-mL Eppendorf tube and the final mass after centrifugation. Then, the mass of the plasma sample was converted to recovered volume by using the average density of plasma (1.025 g/mL). The total theoretical plasma volume was determined based on the measured hematocrit value and input sample volume. Separation efficiency was defined as the ratio of collected plasma volume to total theoretical plasma volume.

### Recovery of Total Protein

Recovery was calculated as the ratio of total protein in plasma samples obtained from the plasma separation device to the concentration of total protein in plasma samples obtained via centrifugation. The Pierce 660 nm protein assay was used to quantify the total protein in plasma samples according to an established protocol. Briefly, 150 μL of the Pierce 660 reagent was added into a microwell plate, followed by 10 μL of diluted plasma (1:100 in 1×PBS). The microwell plate was incubated for 5 minutes at room temperature before reading at 660 nm using a Varioskan LUX microplate reader. A calibration curve was prepared using BSA solutions over a linear range from 0.05-2 mg/mL.

Recovery of High Abundance Protein h-IgG

Bio-Layer Inteferometry (K2 Octet, Pall Fortébio) was used to quantitate human immunoglobulin G (h-IgG) in reference plasma (i.e., obtained via centrifugation) and recovered plasma samples (i.e., obtained from the plasma separation device). A 96-well plate format with fiber-optic biosensors coated with Protein-A was used to measure the binding rate of h-TgG to Protein-A. Calibration curves were prepared using polyclonal h-IgG standards of known concentrations, ranging from 1-700 μg/mL (Pall Fortébio). The plasma samples were diluted 1:1000 in 1×Kinetics Buffer (Pall Fortébio) before quantitation to ensure the signal fell within the working range of the calibration curve.

The calibration curves were fit using a linear-point-to-point method, as described in the Protein-A Biosensor data sheet. The linear model was used to determine unknown concentrations for the reference plasma (N=20) and the recovered plasma (N=20). The two groups were statistically analyzed using a two-tailed Student's t-test with equal variances.

### Evaluation of Purity for Collected Plasma

The concentration of hemoglobin in recovered plasma was quantified to evaluate the purity of samples obtained by the plasma separation device. Extent of hemolysis was defined as the ratio of hemoglobin in plasma to total hemoglobin quantified according to an established method. For quantification of total hemoglobin in whole blood samples, a ratio of 1:250 was used (e.g., 4 μL of whole blood to 1 mL Drabkin's reagent containing 0.05% (v/v) Brij 25). Calibration curves were prepared daily using lyophilized hemoglobin standard rehydrated with diH2O (18 MΩ) and diluted over a range 3-20 g/dL. For quantification of hemoglobin in plasma samples, a ratio of 1:10 was used (e.g., 20 μL of whole blood to 0.2 mL Drabkin's reagent containing 0.05% (v/v) Brij 25). Calibration curves were prepared daily using lyophilized hemoglobin standard rehydrated with diH2O (18 MΩ) and diluted over a range 0.09-3 g/dL. The mixture was incubated at room temperature (i.e., 21° C.) for 15 minutes and absorbance was measured at 540 nm using a Varioskan LUX microplate reader. Plasma samples were collected from each plasma separation device and hemoglobin was quantified to determine extent of hemolysis against total hemoglobin concentration in whole blood. The LOD for both assays (i.e., 1:250 and 1:10 dilutions) were calculated using purified plasma obtained via centrifugation from three different donors.

### Tetanus Lateral Flow Test

Utility of the plasma separation device was demonstrated by applying collected plasma directly to commercially available lateral flow tests. All blood samples were collected from donors that had been vaccinated against tetanus and therefore contained tetanus antibodies. Positive controls from whole blood were prepared via centrifugation and negative controls from assay buffer provided with the lateral flow tests. 20 μL of plasma were applied to the sample input zone on the lateral flow test using a micropipette. 3 drops of assay buffer were then immediately added onto the sample input zone and 10 minutes were allowed to pass before scanning the results with an Epson V600 Perfection flatbed scanner at 800 DPI.

### Results and Discussion

### Selection of Absorbent Materials (e.g., Third Layer)

A source of capillarity facilitated the performance of passive separation of plasma from whole blood. Capillarity was provided by the absorbent material, which was in direct contact with the separation materials above (FIG. 2). The desired material would provide (i) a fast wicking rate, (ii) high absorbency, and (iii) quantitative release of absorbed liquid. Three different wicking materials were tested: cellulose, polyester, and a rayon/polypropylene blend. The cellulose material had the lowest absorbency (65.0±7.0 μL/cm²) and released only 19% of the absorbed liquid (Table 2). In stark contrast, both the polyester and rayon/polypropylene blend materials absorbed 587.0±40.1 μL/cm² and 393.7±23.6 μL/cm², respectively. These super absorbent materials also yielded high percentages for the release of absorbed liquid at 93% (polyester) and 84% (rayon/polypropylene blend).

TABLE 2
Performance of various absorbent materials. Values represent the
average of five replicates and standard error of the mean.

| | Absorbency | Volume Recovery | |
|---|---|---|---|
| Material | (μL/cm²) | (μL/cm²) | Release |
| Cellulose | 65 ± 7 | 12 ± 7 | 19% |
| Polyester | 587 ± 40 | 544 ± 22 | 93% |
| Rayon/Polypropylene Blend | 393 ± 23 | 330 ± 27 | 84% |

Both the polyester and rayon/polypropylene blend materials in the device were evaluated for wicking ability in conjunction with the PSM. While the polyester material was more absorbent than the rayon/propylene blend, it caused more hemolysis of the blood sample. The rayon/polypropylene blend material did not cause hemolysis and therefore provided a better wicking source for separating plasma from whole blood in the device.

### Baseline Performance of the PSM (e.g., Second Layer)

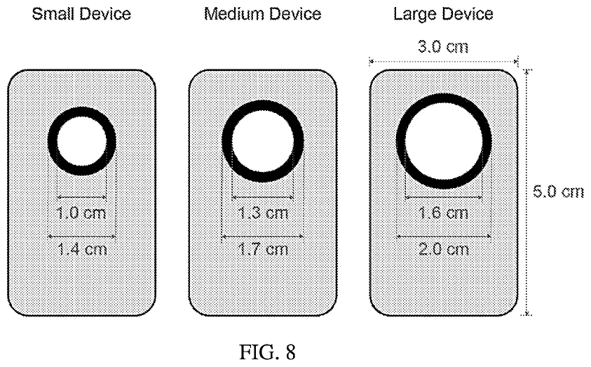

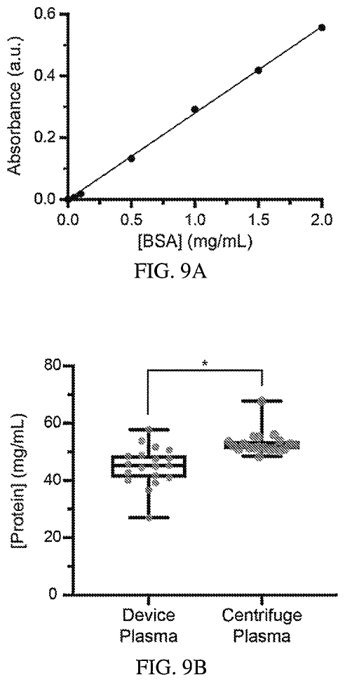

Three devices of different sizes (FIG. 8) were designed and tested with whole blood to establish baseline separation efficiencies using only a single layer of PSM. As shown in FIG. 8, the inner black ring on each device was the cavity ledge of acrylic (half depth cut, 0.317 cm), which provided physical support for the separation materials. The inner white circle was the open region of the device (full depth cut, 0.635 cm), which allowed direct contact between the absorbent material and the separation materials. The area of the plasma separation membrane determined the allowable sample input volume according to the manufacturer (40-50 μL cm-1). Theoretical sample input volumes were calculated for each device based on the minimum and maximum loading capacities for Vivid GR plasma separation membrane from Pall Corp (Table 3).

TABLE 3
Theoretical sample input volumes (μL) for small, medium, and
large devices.

| Plasma Separation | Theoretical Sample Input Volume (μL) | | |
|---|---|---|---|
| Membrane | Small Device | Medium Device | Large Device |
| Loading Capacity | (1.0 cm | (1.3 cm | (1.6 cm |
| (μL cm⁻¹) | diameter) | diameter) | diameter) |
| Minimum | 31.4 | 53.1 | 80.4 |
| Maximum | 39.3 | 66.3 | 100.5 |

A variety of PSMs were tested and the material with the greatest loading capacity (i.e., input volume of blood per area) and consistency was identified. Baseline separation for each device was measured after 10 minutes following sample addition and yielded a consistent volume range of 16-20% (maximum of 27.5 μL) of available plasma from a 250 μL sample input (Table 4). Minimal hemolysis was observed in all three devices (Table 4). The large device (1.6 cm diameter) achieved a higher degree of separation than the smaller devices under these conditions.

TABLE 4
Baseline data for each device with no pre-filter material. Sample input
volumes were 250 μL and 150 μL whole blood. Separation time (10 minutes) and
hematocrit (ca. 45%) were constant.

| | 250 μL | | | | 150 μL | | | |
|---|---|---|---|---|---|---|---|---|
| | Average Recovered | | Separation | Extent of | Average Recovered | | Separation | Extent of |
| Device | Volume (μL) | SEM | Efficiency | Hemolysis | Volume (μL) | SEM | Efficiency | Hemolysis |
| Small | 22.5 | 1.4 | 16.6% | 2.4% | 23.5 | 1.7 | 28.9% | 2.5% |
| (1.0 cm diameter) | | | | | | | | |
| Medium | | | | | | | | |
| (1.3 cm diameter) | 22.5 | 0.9 | 16.6% | 1.9% | 21.7 | 0.8 | 26.9% | 3.9% |
| Large | 27.1 | 1.9 | 20.0% | 2.7% | 22.3 | 2.1 | 27.6 | 2.5% |
| (1.6 cm diameter) | | | | | | | | |

The decreased separation of the smaller 250 μL devices (1.0 cm diameter) was attributed to an excess number of RBCs that clogged the pores of the PSM and impeded the flow of plasma through the membrane to the absorbent material below. In order to alleviate this burden on the PSM, a pre-filter material was included to remove RBCs and allow the plasma to flow through the membrane for collection. Potential pre-filter materials included fiberglass, polyester mesh with pore sizes ranging from 1-11 μm, and a fibrous membrane for the isolation of leukocytes from whole blood (Leukosorb, Pall Corp).

### Material Screen for Pre-Filter (e.g., First Layer)

Fiberglass (Ahlstrom grade 8950) was initially selected for its propensity to act as a chromatographic material for blood separation without binding proteins or causing hemolysis. However, a single layer of fiberglass actually decreased the separation efficiency of the device by 3.5% (Table 6). The fiberglass was 0.25 mm thick with a reported void volume of 46 μL/cm². While fiberglass was capable of separating plasma from whole blood, the wicking rate and void volume of the material negatively impacted the performance of the device and required separation times in excess of 90 minutes.

The fibers of the polyester mesh did not absorb fluids or swell when in contact with liquid samples. This effectively lowered the void volume of the material, which increased the total recovery of plasma in the device. RBCs have an average size distribution of 6-8 μm and a biconcave disc geometry. However, since RBCs are easily deformable, a range of pore sizes were studied in an effort to create a pre-filter based on size exclusion for capturing RBCs. Initially, multiple layers of mesh with a pore size of 1 μm were tested as a pre-filter in a large plasma separation device (Table 5).

TABLE 5
Performance of multiple layers of mesh as pre-filter in large acrylic
devices. Sample input volume (150 μL whole blood) and
separation time (10 minutes) were constant.

| Large | Average Recovered | | Separation | Extent of |
|---|---|---|---|---|
| Acrylic Device | Volume (μL) | SEM | Efficiency | Hemolysis |
| Baseline | 22.3 | 2.1 | 27.6% | 2.5% |
| 1 layer | 30.0 | 2.0 | 36.8% | 1.3% |
| 2 layers | 28.6 | 1.5 | 35.1% | 3.2% |

In another embodiment, a mesh with a pore size of 11 μm was used to remove larger cells such as leukocytes (average diameter of 7-20 μm) from the sample matrix upon initiation of the device. The next layer had a pore size of 6 μm to remove any remaining leukocytes as well as a portion of RBCs. To ensure removal of all RBCs from the sample matrix prior to reaching the PSM, a final layer of polyester mesh with pore size of 1 μm was included. This construct of meshes acted as an effective pre-filter by increasing the separation efficiency by 9.6% and decreasing the extent of hemolysis by 1.2% within 10 minutes (Table 6). Iterations of this construct were investigated with single layers of polyester mesh (e.g., 1 μm, 6 μm, 11 μm), which yielded similar results. A maximum of 33.6% separation efficiency was achieved using two layers of polyester mesh with 1 and 6 μm pore sizes (Table 6).

TABLE 6
Performance of various pre-filter materials. Sample
input volume (250 μL whole blood), separation time
(10 minutes), and hematocrit (ca. 45%) were constant.

| | Average | | | |
|---|---|---|---|---|
| | Recovered | | Separation | Extent of |
| Pre-Filter Material | Volume (μL) | SEM | Efficiency | Hemolysis |
| Polyester Mesh (1 μm) | 37.3 | 0.7 | 27.5% | 1.7% |
| Polyester Mesh (6 μm) | 43.8 | 1.2 | 32.3% | 1.7% |
| Polyester Mesh (11 μm) | 45.3 | 0.6 | 33.4% | 1.5% |
| Polyester Mesh (1 + 6 μm) | 45.5 | 2.8 | 33.6% | 1.5% |
| Polyester Mesh (1 + | 40.1 | 1.8 | 29.6% | 1.5% |
| 6 + 11 μm) | | | | |
| Fiberglass | 22.4 | 5.0 | 16.5% | 3.8% |
| Leukosorb | 70.6 | 2.6 | 51.1% | 4.3% |

To complement the function of commercially available PSM for passively separating plasma from the complex matrix of whole blood, a fibrous membrane (Leukosorb by Pall Corp.) was used. Initial screening of this material yielded 51.1% separation efficiency and an average recovered volume of 70.6 μL of pure plasma (Table 6). Coupling the PSM and Leukosorb pre-filter allowed a high degree of separation of plasma from whole blood. To further characterize the performance of this device format, each parameter was optimized using the large device (1.6 cm diameter) to obtain the largest volume of plasma from the sample of whole blood.

### Device Optimization Using a Leukosorb First Layer and a PSM Second Layer

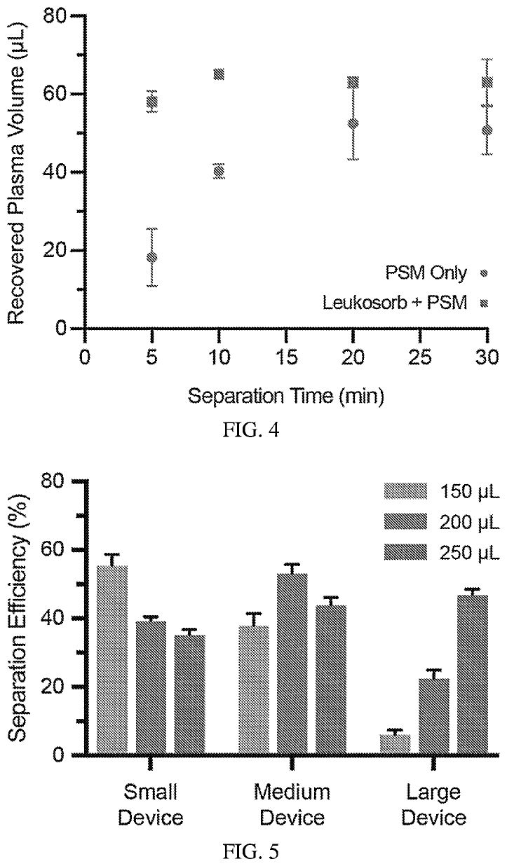

The combined theoretical void volume of the PSM (ca. 20 μL/cm²) and Leukosorb (ca. 40-70 μL/cm2) pre-filter with 1.6 cm diameter was 120-181 μL. The void volume was estimated to be approximately 150 μL by saturating the membranes with water and measuring the mass difference of the dry materials. While this was a considerable volume and directly impacted the maximum achievable separation efficiency, the addition of Leukosorb as a pre-filter increased the separation efficiency of the PSM three-fold after only 5 minutes of separation (FIG. 4).

The use of a Leukosorb prefilter with the PSM was evaluated for a 250 μL sample volume (47% Hct) in the small, medium, and large plasma separation device. The data was collected after 10 minutes were allowed for separation (Table 7).

TABLE 7
Data for small, medium, and large plasma separation device with
Leukosorb first layer and PSM second layer. Separation time
(10 min) and sample volume (250 μL) were constant.

| | Average Recovered | | Separation | Extent of |
|---|---|---|---|---|
| | Volume (μL) | SEM | Efficiency | Hemolysis |
| Large | 53.9 | 2.7 | 39.9% | 3.0% |
| Medium | 45.8 | 4.5 | 33.9% | 6.6% |
| Small | 29.7 | 1.7 | 21.9% | 4.1% |

Allowing separation to continue over a total of 30 minutes showed that Leukosorb and PSM together consistently outperformed PSM on its own. The maximum separation efficiency for PSM with no pre-filter was 35.6% after 20 minutes (Table 8). In contrast, PSM with a single layer of Leukosorb pre-filter yielded 43.5% separation efficiency after only 10 minutes (Table 9). Both device formats exhibited minimal hemolysis (<2.4%) at the maximum separation efficiency.

TABLE 8
Data for a large plasma separation device (1.6 cm diameter)
with no pre-filter material (N = 3).

| | Average Recovered | | Separation | Extent of |
|---|---|---|---|---|
| Separation Time | Volume (μL) | SEM | Efficiency | Hemolysis |
| 5 min | 18.3 | 7.4 | 12.2% | 2.0% |
| 10 min | 40.3 | 1.8 | 26.8% | 2.4% |
| 20 min | 52.5 | 9.2 | 35.6% | 2.4% |
| 30 min | 50.7 | 6.2 | 34.3% | 1.5% |

TABLE 9
Data for large plasma separation device (1.6 cm diameter) with a
single layer of Leukosorb as the pre-filter material (N = 3).

| Separation | Average Recovered | | Separation | Extent of |
|---|---|---|---|---|
| Time | Volume (μL) | SEM | Efficiency | Hemolysis |
| 5 min | 58.1 | 2.6 | 38.8% | 0.8% |
| 10 min | 65.1 | 1.2 | 43.5% | 2.0% |
| 10 min | 72.1 | 2.0 | 48.5% | 3.8% |
| 20 min | 63.1 | 1.4 | 42.0% | 12.5% |
| 30 min | 63.1 | 5.9 | 42.0% | 2.1% |

Testing with Various Hematocrit Values

The number of RBCs in a sample of whole blood could affect both the total plasma yield as well as the plasma quality produced in separation. If the number of RBCs was increased, that could increase the burden on the PSM and result in unwanted hemolysis and sample contamination with intraerythrocytic contents. Therefore, the device was tested with samples of whole blood with varying hematocrit values (see Table 10 and Table 11). The maximum separation efficiency was 53.8% with an average recovered volume of 65.6 μL for a sample of whole blood with a hematocrit of 30% (see Table 11). Varying the hematocrit generally yielded similar values for recovered plasma volume, however, the separation efficiency generally decreased (see Table 11). Decreasing the hematocrit results in an increase in the theoretical volume of available plasma, which affects the value of separation efficiency. While an increase in average recovered volume (72.1 μL) for samples of whole blood was observed at 40% Hct, there was a decrease in average recovered volume (63.0 μL) for samples of whole blood at 35% Hct (Table 10).

TABLE 10
Plasma separation data for the large plasma separation device using
samples of whole blood over a range of hematocrit values (N = 5).

| | Average Recovered | | Separation | Extent of |
|---|---|---|---|---|
| Hematocrit | Volume (μL) | SEM | Efficiency | Hemolysis |
| 45% | 70.6 | 2.6 | 51.1% | 4.3% |
| d40% | 72.1 | 2.0 | 48.5% | 3.7% |
| 35% | 63.0 | 2.5 | 38.8% | 2.9% |

TABLE 11
Plasma separation data for the large plasma separation device
(1.6 cm diameter) using samples of whole blood over a range
of hematocrit values (20-60% Hct, N = 3).

| | Average Recovered | | Separation | Extent of |
|---|---|---|---|---|
| Hematocrit | Volume (μL) | SEM | Efficiency | Hemolysis |
| 20% | 60.6 | 3.3 | 30.1% | 1.9% |
| 30% | 65.6 | 3.9 | 53.8% | 1.0% |
| 40% | 60.4 | 4.8 | 40.6% | 0.9% |
| 50% | 60.0 | 2.5 | 48.0% | 0.9% |
| 60% | 43.9 | 0.8 | 44.7% | 1.1% |

To optimize the separation efficiency achieved by the plasma separation device, each device (small, medium, large) was tested with a range of input sample volumes from 150-250 μL at a constant hematocrit value of 45% (FIG. 5). Each device had a specific input volume that resulted in maximum separation efficiency after 10 minutes of separation with a constant hematocrit (45% Hct). The small device (1.0 cm diameter) produced optimal separation efficiency of 55.5% with a sample input of 150 μL. The medium device (1.3 cm diameter) produced optimal separation efficiency of 53.3% with a sample input of 200 μL. The large device (1.6 cm diameter) produced optimal separation efficiency of 47.0% with a sample input of 250 μL. The corresponding average recovered volume of plasma can be found in Table 12 for each device. Each device consistently showed a decrease in separation efficiency when the input sample volume deviated from the optimal input sample volume.

TABLE 12
Data for plasma separation devices (small, medium, and large)
with a single layer of Leukosorb as the pre-filter (N = 5).

| | | Average | | | |
|---|---|---|---|---|---|
| Sample Input | Device | Recovered | | Separation | Extent of |
| Volume (μL) | Size | Volume (μL) | SEM | Efficiency | Hemolysis |
| 150 | Small | 45.3 | 2.6 | 55.5% | 5.3% |
| | Medium | 30.7 | 2.7 | 38.0% | 0.7% |
| | Large | 5.1 | 1.1 | 6.2% | 0.6% |
| 200 | Small | 42.8 | 1.2 | 39.4% | 4.0% |
| | Medium | 57.3 | 2.7 | 53.3% | 5.4% |
| | Large | 24.8 | 2.5 | 22.6% | 0.6% |
| 250 | Small | 47.8 | 1.8 | 35.4% | 9.4% |
| | Medium | 59.1 | 2.8 | 44.1% | 2.2% |
| | Large | 64.8 | 2.2 | 47.0% | 4.2% |

### Plasma Quality

Pure plasma obtained from standard methods-such as centrifugation-contains various proteins, solutes, and platelets. These include analytes of interest which must be conserved during separation so that the sample is relevant for subsequent analysis and diagnostic utility. Plasma sample impurity may arise from ruptured red blood cells and the release of intraerythrocytic analytes such as hemoglobin. The quality of plasma obtained from the device was evaluated by quantifying (i) total protein, (ii) specific h-IgG (high abundance), and (iii) specific IL-X (low abundance). Purity was measured by quantification of hemoglobin and diagnostic utility was demonstrated by direct application of collected plasma to a commercially available lateral flow test for the tetanus antibody. Whole blood from a single donor was applied to 20 plasma separation devices and a reference sample of pure plasma was prepared via centrifugation.

In order to improve the elution process, data evaluating the protein recovery was collected over varying buffer compositions (Table 13). Once the samples were fully dried, a volume of buffer (containing various surfactants) was added to the absorbent material to rehydrate the analytes found in plasma. Then the sample was extracted from the absorbent material via the same centrifugation method previously described.

TABLE 13
Total protein recovery as a result of varying buffer compositions

| Eluent | [Protein] (g/L) | SEM (g/L) | Protein Recovery |
|---|---|---|---|
| Water | 34.8 | 9.1 | 61% |
| Phosphate Buffered | 44.3 | 0.5 | 91% |
| Saline | | | |
| PBS + Surfactant 1 | 46.5 | 0.8 | 97% |
| PBS + Surfactant 2 | 46.8 | 0.9 | 97% |
| PBS + Surfactant 3 | 46.5 | 0.6 | 97% |
| PBS + Surfactant 4 | 47.1 | 1.0 | 98% |
| PBS + Surfactant 5 | 47.0 | 0.6 | 98% |
| PBS + Surfactant 6 | 48.3 | 0.0 | 101% |

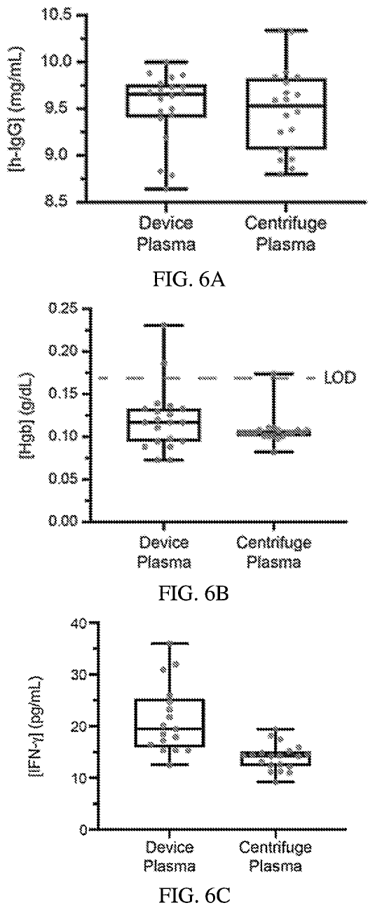

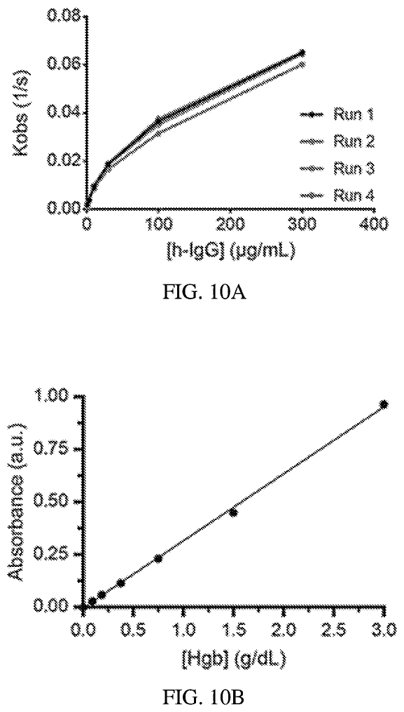

Total protein analysis of plasma obtained from the device yielded a recovery of 86.2% using the Pierce 660 assay (FIGS. 10A-10B). A two-tailed Student's t-test yielded a p-value of <0.0001, indicating a loss of total protein between the two sets of plasma samples. Adsorption of proteins (e.g., albumins and globulins) was expected in porous materials. To better evaluate the loss of proteins in the device, specific proteins of interest were quantified at high (h-IgG) and low (h-IFNT) quantities. The concentration of h-IgG in recovered plasma from the device was nearly identical to the concentration in reference plasma, suggesting that there was no apparent loss of human IgG to the materials in the plasma separation device (FIG. 6A and Table 14).

TABLE 14
Human IgG concentration in reference and recovered plasma as
quantified by Bio-Layer Interferometry. The average
concentrations and standard deviations are nearly identical between
the reference plasma (N = 20) and the recovered plasma (N = 20).

| | Centrifuge Plasma | Device Plasma |
|---|---|---|
| Average [h-IgG] (mg/mL) | 9.48 | 9.52 |
| SD | 0.45 | 0.38 |

A two-tailed Student's t-test yielded a p-value of 0.786, providing no evidence of a difference in h-IgG concentration between the two sets of plasma samples (FIG. 6A).

Purity of the plasma collected with the plasma separation device was verified by quantification of released hemoglobin as a function of hemolysis (FIGS. 10A-10B). The LOD was calculated as 0.17 g/dL hemoglobin using purified plasma (i.e., obtained via centrifugation) from three different donors (FIG. 10B). Both the reference and recovered samples yielded hemoglobin concentrations below the LOD at 0.11±0.02 and 0.12±0.04 g/dL, respectively (FIG. 6B). Low concentrations of released hemoglobin indicated a lack of hemolysis and subsequent high purity of plasma samples obtained from the device.

The amount of the low concentration analyte (pg/mL), IFN-γ, present in the recovered plasma sample was in agreement with that in the reference plasma sample, as shown in FIG. 6C. Quantitation of IFN-γ by qPCR using a ProQuantum immunoassay kit showed no loss of IFN-γ in the recovered plasma sample even at extremely low concentrations, indicating that the quality of the plasma is conserved even for low abundance proteins. A two-tailed Student's t-test yielded a p-value of <0.001 and the difference in average concentrations of IFN-γ between the recovered plasma sample and the reference plasma sample was 7.3 pg/mL, which is within the tolerance of the ProQuantum immunoassay kit.

Additionally, the recovery of HIV RNA in the recovered plasma sample was also evaluated. Simulated samples of HIV-positive whole blood at a viral load of 50,000 copies/mL were prepared by spiking plasma from an HIV-positive patient into whole blood from an HIV-negative patient. RT-qPCR was used to detect and quantify the presence of HIV RNA. All experiments were performed in triplicate. The plasma recovered from the simulated HIV-positive whole blood samples had an average threshold cycle value (Ct, unitless) of 23.3±0.6, while the average Ct for control plasma samples, obtained from the simulated whole blood via centrifugation, was 22.1±0.3. These Ct values correlate to 43.3% elution efficiency for total HIV RNA collected from the recovered plasma sample.

HIV-positive plasma was tested on the device as a less complex sample matrix than whole blood. When HIV-positive plasma was added to the device, a very slight difference in Ct values (24.1 vs 24.8) was observed. The loss of efficiency with whole blood samples was likely due to matrix effects, where some HIV virions were nonspecifically filtered during the plasma separation process due to interactions with the cells contained in the otherwise naïve blood.

### Demonstration of a Diagnostic Test

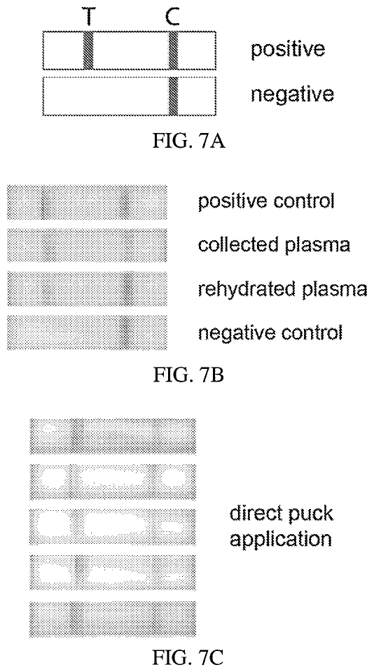

The goal was to produce a device capable of passive plasma separation for use at the point-of-care in resource limited settings. While the majority of these analyses were performed on liquid plasma samples collected via centrifugation following separation in the device, this example also demonstrated the direct utility of the device for performing a lateral flow test without centrifugation (FIG. 7). Samples collected with the plasma separation device were (i) recovered in liquid form using a centrifuge (FIG. 7B), (ii) dried overnight in the absorbent puck, rehydrated with elution buffer, recovered in liquid form using a centrifuge (FIG. 7B), and (iii) directly applied to a lateral flow test without centrifugation (FIG. 7C) (e.g., like a stamp). In agreement with the positive samples obtained by centrifugation (FIG. 7A), positive results were obtained for every condition tested using undiluted human plasma obtained from the device. Slight attenuation of the control line occurred when the sample was directly applied from the absorbent puck (FIG. 7C), however, the diagnostic output of the lateral flow test was unaffected.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
a first layer, wherein the first layer is porous and has a first mode pore size that is greater than or equal to 1 micron and less than or equal to 30 microns;
a second layer having a first surface and a second surface, wherein the second layer is porous and greater than or equal to 20% of the pores of the second layer have a pore size of less than or equal to 20 microns; and
a third layer, wherein the third layer is porous and has an absorbency of greater than or equal to 80 microliters/cm² and less than or equal to 600 microliters/cm²; and
wherein the second layer is positioned between the first layer and the third layer.

2. A method, comprising:
passing a blood sample across a first layer to produce a blood sample with reduced red blood cells,
passing the blood sample with reduced red blood cells across a second layer to produce a blood sample with further reduced red blood cells; and
passing the blood sample with further reduced red blood cells into a third layer that has an absorbency of greater than or equal to 80 microliters/cm² and less than or equal to 500 microliters/cm²;
wherein the first layer, the second layer, and the third layer are porous.

3. The method of claim 2, wherein the first layer has a first mode pore size that is greater than or equal to 1 micron and less than or equal to 30 microns.

4. The method of any one of claims 2-3, wherein the second layer has a first surface and a second surface, and greater than or equal to 20% of the pores of the second layer have a pore size of less than or equal to 20 microns.

5. The method of any one of claims 2-4, wherein an article comprises the first layer, second layer, and third layer.

6. The method of any one of claims 2-5, wherein the second layer is positioned between the first layer and the third layer.

7. The article or method of any one of the preceding claims, wherein the first layer is in direct contact with the second layer and/or the second layer is in direct contact with the third layer.

8. The article or method of any one of the preceding claims, wherein the first mode pore size is greater than or equal to 2 microns and less than or equal to 25 microns.

9. The article or method of any one of the preceding claims, wherein the first mode pore size is greater than or equal to 15 microns and less than or equal to 25 microns.

10. The article or method of any one of the preceding claims, wherein the second layer has a second mode pore size, and the second mode pore size is greater than or equal to 2 microns and less than or equal to 30 microns.

11. The article or method of claim 10, wherein the second mode pore size is greater than or equal to 10 microns and less than or equal to 20 microns.

12. The article or method of any one of the preceding claims, wherein the second layer has a second mode pore size, and the second mode pore size is smaller than the first mode pore size of the first layer.

13. The article or method of any one of the preceding claims, wherein greater than or equal to 50% of the pores of the second layer have a pore size of less than or equal to 20 microns.

14. The article or method of any one of the preceding claims, wherein greater than or equal to 20% of the pores of the second layer have a pore size of less than or equal to 10 microns.

15. The article or method of any one of the preceding claims, wherein the absorbency of the third layer is greater than or equal to 200 microliters/cm² and less than or equal to equal to 450 microliters/cm².

16. The article or method of any one of the preceding claims, wherein the third layer is configured to absorb blood plasma.

17. The article or method of any one of the preceding claims, wherein the third layer has a third mode pore size, and the third mode pore size is greater than or equal to 20 microns and less than or equal to 150 microns.

18. The article or method of any one of the preceding claims, wherein the third layer has a third mode pore size, and the third mode pore size is greater than or equal to 75 microns and less than or equal to 125 microns.

19. The article or method of any one of the preceding claims, wherein the third layer has a release of greater than or equal to 35%.

20. The article or method of any one of the preceding claims, wherein the third layer has a release of greater than or equal to 50%.

21. The article or method of any one of the preceding claims, wherein the third layer has a release of greater than or equal to 70%.

22. The article or method of any one of the preceding claims, wherein the second layer has a gradient in mode pore size between the first surface and the second surface, such that the first surface, which faces the first layer, has a mode pore size, the second surface, which faces the third layer, has a mode pore size, and the mode pore size of the second surface is smaller than the mode pore size of the first surface.

23. The article or method of claim 22, wherein a ratio of the mode pore size of the first surface to the mode pore size of the second surface is greater than or equal to 5:1 and less than or equal to 1,000:1.

24. The article or method of any one of claims 22-23, wherein a ratio of the mode pore size of the first surface to the mode pore size of the second surface is greater than or equal to 100:1 and less than or equal to 200:1.

25. The article or method of any one of the preceding claims, wherein there are no intervening layers between the first layer and the second layer and/or the second layer and the third layer.

26. The article or method of any one of the preceding claims, wherein the first layer is adhered to the second layer and/or the second layer is adhered to the third layer.

27. The article or method of any one of the preceding claims, wherein the first layer is adhered to the second layer with adhesive around the perimeter of the first layer and/or second layer where they are in contact, and wherein the adhesive creates a full seal around the perimeter.

28. The article or method of any one of the preceding claims, wherein the second layer is adhered to the third layer with adhesive around the perimeter of the second layer and/or third layer where they are in contact, and wherein the adhesive creates a full seal around the perimeter.

29. The article or method of any one of claims 27-28, wherein the adhesive has a thickness of greater than or equal to 0.03 millimeters and less than or equal to 0.2 millimeters.

30. The article or method of any one of the preceding claims, wherein the first layer has a thickness of greater than or equal to 150 microns and less than or equal to 500 microns.

31. The article or method of any one of the preceding claims, wherein a maximum horizontal dimension of the first layer, the second layer, and/or the third layer is greater than or equal to 20 millimeters and less than or equal to 500 millimeters.

32. The article or method of any one of the preceding claims, wherein the first layer comprises greater than or equal to 2 sub-layers and less than or equal to 4 sub-layers.

33. The article or method of claim 32, wherein each of the sub-layers has a different mode pore size and the sub-layers are arranged such that a gradient in mode pore size is formed.

34. The article or method of any one of the preceding claims, wherein the first layer, second layer, and third layer are stacked vertically.

35. The article or method of any one of the preceding claims, wherein the first layer comprises fiberglass, polyester, a fibrous membrane, polyether sulfone, polyester, nylon, and/or mesh.

36. The article or method of any one of the preceding claims, wherein the first layer comprises polyester.

37. The article or method of any one of the preceding claims, wherein the second layer comprises a polymer.

38. The article or method of any one of the preceding claims, wherein the second layer comprises polyether sulfone.

39. The article or method of any one of the preceding claims, wherein the second layer comprises a plasma separation membrane.

40. The article or method of any one of the preceding claims, wherein the third layer comprises rayon and/or polyester.

41. The article or method of any one of claims 1-40, wherein the third layer comprises a blend of rayon and polypropylene.

42. The article or method of any one of the preceding claims, wherein the article further comprises a support structure.

43. The article or method of any one of the preceding claims, wherein the article further comprises a support structure comprising a cavity.

44. The article or method of claim 43, wherein a maximum horizontal dimension of the cavity is greater than or equal to the maximum horizontal dimension of the third layer.

45. The article or method of any one of the preceding claims, wherein the support structure comprises a plastic, an acrylic, and/or a metal.

46. The article or method of any one of the preceding claims, where the third layer has a thickness of greater than or equal to 200 microns and less than or equal to 800 microns.

47. The article or method of any one of the preceding claims, where the third layer has a thickness of greater than or equal to 250 microns and less than or equal to 500 microns.

48. The article of method of any one of the preceding claims, wherein the first layer has an absorbency of greater than or equal to 10 microliters/cm² and less than or equal to 100 microliters/cm².

49. The article of method of any one of the preceding claims, wherein the first layer has an absorbency of greater than or equal to 20 microliters/cm² and less than or equal to 50 microliters/cm².

50. The article of method of any one of the preceding claims, wherein the second layer has an absorbency of greater than or equal to 10 microliters/cm² and less than or equal to 50 microliters/cm².

51. The article of method of any one of the preceding claims, wherein the second layer has an absorbency of greater than or equal to 15 microliters/cm² and less than or equal to 25 microliters/cm².

52. The method of any one of claims 2-51, wherein passing the blood sample across the first layer, passing the blood sample with reduced red blood cells across the second layer, and/or passing the blood sample with further reduced red blood cells into the third layer is passive.

53. The method of any one of claims 2-52, wherein the blood sample is undiluted whole blood.

54. The method of any one of claims 2-53, wherein passing the blood sample across the first layer, passing the blood sample with reduced red blood cells across the second layer, and/or passing the blood sample with further reduced red blood cells into the third layer is accomplished within less than or equal to 30 minutes.

55. The method of any one of claims 2-54, wherein passing the blood sample across the first layer, passing the blood sample with reduced red blood cells across the second layer, and/or passing the blood sample with further reduced red blood cells into the third layer is accomplished within less than or equal to 10 minutes.

56. The method of any one of claims 2-55, wherein passing the blood sample across the first layer, passing the blood sample with reduced red blood cells across the second layer, and/or passing the blood sample with further reduced red blood cells into the third layer is accomplished within less than or equal to 5 minutes.

57. The method of any one of claims 2-56, wherein the blood sample has a volume of greater than or equal to 25 microliters.

58. The method of claim 57, wherein the volume is greater than or equal to 100 microliters.

59. The method of any one of claims 2-58, wherein the blood sample has a volume of less than or equal to 500 microliters.

60. The method of any one of claims 2-59, further comprising removing the third layer from the article.

61. The method of any one of claims 2-60, wherein the blood sample with further reduced red blood cells is used directly from the third layer.

62. The method of any one of claims 2-61, wherein the blood sample with further reduced red blood cells is stored inside the third layer.

63. The method of any one of claims 2-62, wherein the blood sample with further reduced red blood cells is stored inside the third layer for greater than or equal to 1 week.

64. The method of any one of claims 2-63, wherein the blood sample with further reduced red blood cells is stored inside the third layer in a dry state.

65. The method of claim 64, wherein the third layer in the dry state is rehydrated.

66. The method of any one of claims 2-65, wherein the blood sample with further reduced red blood cells is collected from the third layer.

67. The method of any one of claims 2-66, wherein the blood sample with further reduced red blood cells is collected from the third layer via centrifuge and/or compression.

68. The method of any one of claims 2-67, wherein the blood sample with further reduced red blood cells is collected from the third layer via centrifugation for less than or equal to 30 minutes.

69. The method of any one of claims 2-68, wherein the blood sample with further reduced red blood cells is collected from the third layer via centrifugation for less than or equal to 10 minutes.

70. The method of any one of claims 2-69, wherein a separation efficiency is greater than or equal to 10%.

71. The method of any one of claims 2-70, wherein a separation efficiency is greater than or equal to 30%.

72. The method of any one of claims 2-71, wherein a separation efficiency is greater than or equal to 50%.

73. The method of any one of claims 2-72, wherein the blood sample with further reduced red blood cells has less than or equal to 15% hemolysis.

74. The method of any one of claims 2-73, wherein the blood sample with further reduced red blood cells has less than or equal to 7% hemolysis.

75. The method of any one of claims 2-74, wherein the blood sample with further reduced red blood cells is used in a clinical test, diagnostic health test, clinical chemistry assay, immunoassay, immunochromatographic assay for antibodies, quantification of cytokines, amplification of viral RNA, rapid dipstick test, HIV viral load assay, cholesterol test, metabolite panel, serology for an infectious disease, and/or therapeutic drug monitoring.

76. The method of any one of claims 2-75, wherein the blood sample with further reduced red blood cells has less than or equal to 5% of the red blood cells in the blood sample.

77. The method of any one of claims 2-76, wherein the blood sample with further reduced red blood cells has less than or equal to 2% of the red blood cells in the blood sample.

78. The method of any one of claims 2-77, wherein the blood sample with further reduced red blood cells has less than or equal to 5% of the white blood cells in the blood sample.

79. The method of any one of claims 2-78, wherein the blood sample with further reduced red blood cells has less than or equal to 2% of the white blood cells in the blood sample.

80. The method of any one of claims 2-79, wherein the volume of the blood sample with further reduced red blood cells passed into the third layer is greater than or equal to 20 microliters and less than or equal to 150 microliters.

81. The method of any one of claims 2-80, wherein the volume of the blood sample with further reduced red blood cells passed into the third layer is greater than or equal to 30 microliters and less than or equal to 150 microliters.

82. The method of any one of claims 2-81, wherein the volume of the blood sample with further reduced red blood cells passed into the third layer is greater than or equal to 50 microliters and less than or equal to 150 microliters.

83. The method of any one of claims 66-82, wherein the volume of the blood sample with further reduced red blood cells collected from the third layer is greater than or equal to 20 microliters and less than or equal to 150 microliters.

84. The method of any one of claims 66-83, wherein the volume of the blood sample with further reduced red blood cells collected from the third layer is greater than or equal to 30 microliters and less than or equal to 150 microliters.

85. The method of any one of claims 66-84, wherein the volume of the blood sample with further reduced red blood cells collected from the third layer is greater than or equal to 50 microliters and less than or equal to 150 microliters.

86. The method of any one of claims 2-85, wherein the blood with further reduced red blood cells has a level of an analyte of interest that is greater than or equal to 40% and less than or equal to 100% a level of the analyte of interest in the blood sample.

87. The method of any one of claims 2-86, wherein the blood with further reduced red blood cells has a level of an analyte of interest that is greater than or equal to 80% and less than or equal to 100% a level of the analyte of interest in the blood sample.

88. The method of any one of claims 86-87, wherein the analyte of interest comprises proteins, nucleic acids, pathogens, metabolites, hormones, electrolytes, lipids, and/or small molecules.
